(19) United States
(12) Patent Application Publication
(10) Pub. No.: US 2021/0292415 A1
(43) Pub. Date: Sep. 23, 2021

(54) METHODS OF TREATING A TUMOR

(71) Applicants: Bristol-Myers Squibb Company, Princeton, NJ (US); Nektar Therapeutics, San Francisco, CA (US)

(72) Inventors: Wendy L. CLEMENS, Perkasie, PA (US); Jonathan ZALEVSKY, Orinda, CA (US); Ute HOCH, San Francisco, CA (US); Mary TAGLIAFERRI, San Anselmo, CA (US)

(73) Assignees: Bristol-Myers Squibb Company, Princeton, NJ (US); Nektar Therapeutics, San Francisco, CA (US)

(21) Appl. No.: 16/761,771

(22) PCT Filed: Nov. 6, 2018
(86) PCT No.: PCT/US2018/059448

§ 371 Date: May 5, 2020

Related U.S. Application Data
(60) Provisional application No. 62/712,814, filed on Jul. 31, 2018.
(60) Provisional application No. 62/629,481, filed on Feb. 12, 2018.
(60) Provisional application No. 62/582,174, filed on Nov. 6, 2017.

(51) Int. Cl. C07K 16/28; C07K 14/715

(52) U.S. Cl. CPC C07K 16/2818; C07K 14/7155; A61K 2039/507; C07K 2317/76; C07K 2317/21

(57) ABSTRACT

This disclosure provides methods for treating a tumor in a subject comprising administering to the subject an anti-PD-1 antibody and a CD-122-biased agonist. In some embodiments, the tumor is derived from a melanoma, a renal cell carcinoma (RCC), a non-small cell lung carcinoma (NSCLC), a urothelial cancer (UC), a breast cancer, or any combination thereof.

### FIELD OF THE DISCLOSURE

This disclosure relates to methods for treating a tumor in a subject comprising administering to the subject an anti-Programmed Death-1 (PD-1) antibody and a CD-122-biased agonist. In some embodiments, the tumor is derived from a melanoma, renal cell carcinoma (RCC), non-small cell lung cancer (NSCLC), a urothelial cancer (UC), a breast cancer, or any combination thereof.

### BACKGROUND OF THE DISCLOSURE

Human cancers harbor numerous genetic and epigenetic alterations, generating neoantigens potentially recognizable by the immune system (Sjoblom et al., (2006) Science 314:268-74). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities.

Targeted therapy of multiple non-redundant molecular pathways regulating immune responses can enhance antitumor immunotherapy. However, not all combinations have acceptable safety and/or efficacy. There remains a need for combination therapies with an acceptable safety profile and high efficacy that enhance antitumor immune responses compared to monotherapy and other immunotherapy combinations.

### SUMMARY OF THE DISCLOSURE

Certain aspects of the present disclosure are directed to a method of treating a subject afflicted with a tumor comprising administering to the subject: (a) an antibody that binds specifically to a Programmed Death-1 (PD-1) receptor and inhibits PD-1 activity (“anti-PD-1 antibody”); and (b) a CD-122-biased agonist. In some embodiments, the CD-122-biased agonist comprises an interleukin-2 (IL-2) protein conjugated to a polymer. In some embodiments, the tumor is derived from a melanoma, a renal cell carcinoma (RCC), a non-small cell lung carcinoma (NSCLC), a urothelial cancer (UC), a breast cancer, or any combination thereof. In some embodiments, the breast cancer is a triple negative breast cancer (TNBC).

Some aspects of the present disclosure are directed to a method of treating a subject afflicted with a tumor derived from a melanoma comprising administering to the subject: (a) an antibody that binds specifically to a Programmed Death-1 (PD-1) receptor and inhibits PD-1 activity (“anti-PD-1 antibody”); and (b) a CD-122-biased agonist. Other aspects are directed to a method of treating a subject afflicted with a tumor derived from a renal cell carcinoma (RCC) comprising administering to the subject: (a) an antibody that binds specifically to a Programmed Death-1 (PD-1) receptor and inhibits PD-1 activity (“anti-PD-1 antibody”); and (b) a CD-122-biased agonist. Other aspects are directed to a method of treating a subject afflicted with a tumor derived from a non-small cell lung carcinoma (NSCLC) comprising administering to the subject: (a) an antibody that binds specifically to a Programmed Death-1 (PD-1) receptor and inhibits PD-1 activity (“anti-PD-1 antibody”); and (b) a CD-122-biased agonist. Other aspects are directed to a method of treating a subject afflicted with a tumor derived from a urothelial cancer (UC) comprising administering to the subject: (a) an antibody that binds specifically to a Programmed Death-1 (PD-1) receptor and inhibits PD-1 activity (“anti-PD-1 antibody”); and (b) a CD-122-biased agonist. Other aspects are directed to a method of treating a subject afflicted with a tumor derived from a triple negative breast cancer (TNBC) comprising administering to the subject: (a) an antibody that binds specifically to a Programmed Death-1 (PD-1) receptor and inhibits PD-1 activity (“anti-PD-1 antibody”); and (b) a CD-122-biased agonist. In some embodiments, the CD-122-biased agonist comprises an interleukin-2 protein conjugated to a polymer. In some embodiments, the polymer comprises a water-soluble polymer. In some embodiments, the polymer is a water-soluble polymer.

In some embodiments, the administering treats the tumor. In some embodiments, the CD-122-biased agonist interacts with an interleukin-2 receptor βγ (IL-2Rβγ) on the surface of a cell. In some embodiments, the CD-122-biased agonist interacts more strongly with an IL-2Rβγ on the surface of a cell than the CD-122-biased agonist interacts with an IL-2Rαβγ on the surface of the cell. In some embodiments, the cell is selected from the group consisting of a natural killer (NK) cell, a CD4+ cell, a CD8+ cell, and any combination thereof. In some embodiments, the CD-122-biased agonist promotes clonal expansion of NK cells, CD8+ cells, CD4+ helper T cells, or any combination thereof. In some embodiments, the CD-122-biased agonist does not promote clonal expansion of CD4+ Treg cells.

In some embodiments, the CD-122-biased agonist comprises the following formula:

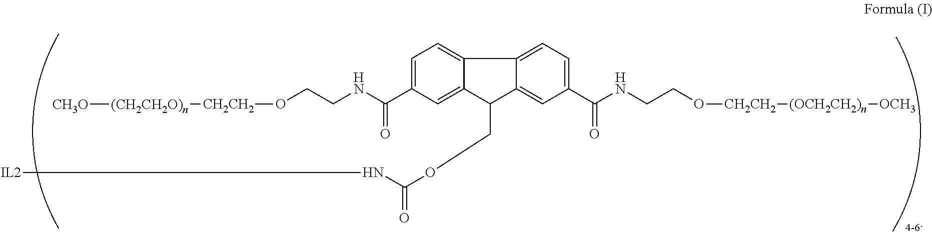

In some embodiments, the administration of the CD-122-biased agonist increases proliferation of tumor infiltrating lymphocytes (TILs) in the tumor as compared to the proliferation of TILs in the tumor prior to the administration. In some embodiments, the administration of the CD-122-biased agonist increases PD-1 expression on effector T cells in the subject as compared to the PD-1 expression on effector T cells prior to the administration.

In some embodiments, the anti-PD-1 antibody cross-competes with nivolumab for binding to human PD-1. In some embodiments, the anti-PD-1 antibody binds to the same epitope as nivolumab. In some embodiments, the anti-PD-1 antibody is a chimeric, humanized or human monoclonal antibody or a portion thereof. In some embodiments, the anti-PD-1 antibody comprises a heavy chain constant region which is of a human IgG1 or IgG4 isotype. In some embodiments, the anti-PD-1 antibody is nivolumab. In some embodiments, the anti-PD-1 antibody is pembrolizumab. In certain embodiments, the anti-PD-1 antibody is OPDIVO®.

In some embodiments, the anti-PD-1 antibody is administered at a flat dose. In some embodiments, the anti-PD-1 antibody is administered at a flat dose of at least about 200, at least about 220, at least about 240, at least about 260, at least about 280, at least about 300, at least about 320, at least about 340, at least about 360, at least about 380, at least about 400, at least about 420, at least about 440, at least about 460, at least about 480, at least about 500 or at least about 550 mg. In some embodiments, the anti-PD-1 antibody is administered at a flat dose ranging from at least about 200 mg to at least about 600 mg. In some embodiments, the anti-PD-1 antibody is administered at a flat dose of about 240 mg, about 360 mg, about 480 mg, or about 560 mg. In some embodiments, the anti-PD-1 antibody is administered at a flat dose of about 240 mg. In some embodiments, the anti-PD-1 antibody is administered at a flat dose of about 360 mg.

In some embodiments, the anti-PD-1 antibody is administered once about every 1, 2, 3, or 4 weeks. In some embodiments, the anti-PD-1 antibody is administered at a flat dose of about 240 mg, about 360 mg, about 480 mg, or about 560 mg about once every 2 weeks or every 3 weeks. In some embodiments, the anti-PD-1 antibody is administered at a flat dose of about 240 mg about once every 2 weeks. In some embodiments, the anti-PD-1 antibody is administered at a flat dose of about 360 mg about once every 3 weeks. In some embodiments, the anti-PD-1 antibody is administered for as long as clinical benefit is observed or until unmanageable toxicity or disease progression occurs.

In some embodiments, the CD-122-biased agonist is administered at a dose ranging from at least about 0.0001 mg/kg to at least about 0.1 mg/kg body weight. In some embodiments, the CD-122-biased agonist is administered at a dose ranging from at least about 0.001 mg/kg to at least about 0.01 mg/kg body weight. In some embodiments, the CD-122-biased agonist is administered at a dose of about 0.003 mg/kg, about 0.004 mg/kg, about 0.005 mg/kg, about 0.006 mg/kg, about 0.007 mg/kg, about 0.008 mg/kg, about 0.009 mg/kg, or about 0.01 mg/kg body weight. In some embodiments, the CD-122-biased agonist is administered at a dose of about 0.003 mg/kg body weight. In some embodiments, the CD-122-biased agonist is administered at a dose of about 0.006 mg/kg body weight. In some embodiments, the CD-122-biased agonist is administered once about every 1, 2, 3, or 4 weeks. In some embodiments, the CD-122-biased agonist is administered at a dose of about 0.003 mg/kg body weight about every 2 weeks. In some embodiments, the CD-122-biased agonist is administered at a dose of about 0.006 mg/kg body weight about every 2 weeks. In some embodiments, the CD-122-biased agonist is administered at a dose of about 0.006 mg/kg body weight about every 3 weeks. In some embodiments, the anti-PD-1 antibody is administered at a dose of about 360 mg every 3 weeks and the CD-122-biased agonist is administered at a dose of about 0.006 mg/kg body weight about every 3 weeks.

In some embodiments, the anti-PD-1 antibody and the CD-122-biased agonist are formulated for intravenous administration.

In some embodiments, the anti-PD-1 antibody and the CD-122-biased agonist are administered sequentially. In some embodiments, the anti-PD-1 antibody and the CD-122-biased agonist are administered within 30 minutes of each other. In some embodiments, the anti-PD-1 antibody is administered before the CD-122-biased agonist. In some embodiments, the CD-122-biased agonist is administered before the anti-PD-1 antibody.

In some embodiments, the anti-PD-1 antibody and the CD-122-biased agonist are administered concurrently in separate compositions.

In some embodiments, the anti-PD-1 antibody and the CD-122-biased agonist are admixed as a single composition for concurrent administration.

In some embodiments, the anti-PD-1 antibody is administered at a subtherapeutic dose. In some embodiments, the CD-122-biased agonist is administered at a subtherapeutic dose. In some embodiments, the anti-PD-1 antibody and the CD-122-biased agonist are each administered at a subtherapeutic dose.

In some embodiments, the tumor comprises one or more cells that express PD-L1, PD-L2, or both.

In some embodiments, the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration.

In some embodiments, the administration of the anti-PD-1 antibody and the CD-122-biased agonist reduces the size of the tumor relative to the size of the tumor prior to the administration. In some embodiments, the size of the tumor is reduced by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% as compared to the size of the tumor prior to the administration.

In some embodiments, the subject received at least one prior chemotherapy treatment.

Other aspects of the present disclosure are directed to a kit for treating a subject afflicted with a cancer, the kit comprising: (a) a dosage ranging from about 10 mg to about 600 mg of an anti-PD-1 antibody; (b) a dosage ranging from about 0.0001 mg to about 0.1 mg of a CD-122-biased agonist; (c) instructions for using the anti-PD-1 antibody and the CD-122-biased agonist in any method disclosed herein.

### Embodiments

E1. A method of treating a subject afflicted with a tumor comprising administering to the subject: (a) an antibody that binds specifically to a Programmed Death-1 (PD-1) receptor and inhibits PD-1 activity (“anti-PD-1 antibody”); and (b) a CD-122-biased agonist.

E2. The method of E1, wherein the CD-122-biased agonist comprises an interleukin-2 (IL-2) protein conjugated to a polymer.

E3. The method of E1 or E2, wherein the tumor is derived from a melanoma, a renal cell carcinoma (RCC), a non-small cell lung carcinoma (NSCLC), a urothelial cancer (UC), a breast cancer, or any combination thereof.

E4. The method of E3, wherein the breast cancer is a triple negative breast cancer (TNBC).

E5. A method of treating a subject afflicted with a tumor derived from a melanoma comprising administering to the subject: (a) an antibody that binds specifically to a Programmed Death-1 (PD-1) receptor and inhibits PD-1 activity (“anti-PD-1 antibody”); and (b) a CD-122-biased agonist.

E6. A method of treating a subject afflicted with a tumor derived from a renal cell carcinoma (RCC) comprising administering to the subject: (a) an antibody that binds specifically to a Programmed Death-1 (PD-1) receptor and inhibits PD-1 activity (“anti-PD-1 antibody”); and (b) a CD-122-biased agonist.

E7. A method of treating a subject afflicted with a tumor derived from a non-small cell lung carcinoma (NSCLC) comprising administering to the subject: (a) an antibody that binds specifically to a Programmed Death-1 (PD-1) receptor and inhibits PD-1 activity (“anti-PD-1 antibody”); and (b) a CD-122-biased agonist.

E8. A method of treating a subject afflicted with a tumor derived from a urothelial cancer (UC) comprising administering to the subject: (a) an antibody that binds specifically to a Programmed Death-1 (PD-1) receptor and inhibits PD-1 activity (“anti-PD-1 antibody”); and (b) a CD-122-biased agonist.

E9. A method of treating a subject afflicted with a tumor derived from a triple negative breast cancer (TNBC) comprising administering to the subject: (a) an antibody that binds specifically to a Programmed Death-1 (PD-1) receptor and inhibits PD-1 activity (“anti-PD-1 antibody”); and (b) a CD-122-biased agonist.

E10. The method of any one of E5 to E9, wherein the CD-122-biased agonist comprises an interleukin-2 protein conjugated to a polymer.

E11. The method of any one of E1 to E10, wherein the administering treats the tumor.

E12. The method of any one of E1 to 11, wherein the CD-122-biased agonist interacts with an interleukin-2 receptor βγ (IL-2Rβγ) on the surface of a cell.

E13. The method of any one of E1 to E12, wherein the CD-122-biased agonist interacts more strongly with an IL-2Rβγ on the surface of a cell than the CD-122-biased agonist interacts with an IL-2Rαβγ on the surface of the cell.

E14. The method of E12 or E13, wherein the cell is selected from the group consisting of a natural killer (NK) cell, a CD4⁺ cell, a CD8⁺ cell, and any combination thereof.

E15. The method of any one of E1 to E14, wherein the CD-122-biased agonist promotes clonal expansion of NK cells, CD8⁺ cells, CD4⁺ helper T cells, or any combination thereof.

E16. The method of E15, wherein the CD-122-biased agonist does not promote clonal expansion of CD4⁺ Treg cells.

E17. The method of any one of E1 to E16, wherein the CD-122-biased agonist comprises the following formula:

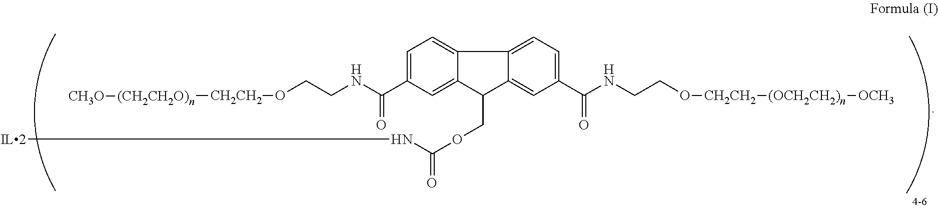

E18. The method of any one of E1 to E17, wherein the administration of the CD-122-biased agonist increases proliferation of tumor infiltrating lymphocytes (TILs) in the tumor as compared to the proliferation of TILs in the tumor prior to the administration.

E19. The method of any one of E1 to E18, wherein the administration of the CD-122-biased agonist increases PD-1 expression on effector T cells in the subject as compared to the PD-1 expression on effector T cells prior to the administration.

E20. The method of any one of E1 to E19, wherein the anti-PD-1 antibody cross-competes with nivolumab for binding to human PD-1.

E21. The method of any one of E1 to E20, wherein the anti-PD-1 antibody binds to the same epitope as nivolumab.

E22. The method of any one of E1 to E21, wherein the anti-PD-1 antibody is a chimeric, humanized or human monoclonal antibody or a portion thereof.

E23. The method of any one of E1 to E22, wherein the anti-PD-1 antibody comprises a heavy chain constant region which is of a human IgG1 or IgG4 isotype.

E24. The method of any one of E1 to E23, wherein the anti-PD-1 antibody is nivolumab.

E25. The method of any one of E1 to E24, wherein the anti-PD-1 antibody is pembrolizumab.

E26. The method of any one of E1 to E25, wherein the anti-PD-1 antibody is administered at a flat dose.

E27. The method of any one of E1 to E26, wherein the anti-PD-1 antibody is administered at a flat dose of at least about 200, at least about 220, at least about 240, at least about 260, at least about 280, at least about 300, at least about 320, at least about 340, at least about 360, at least about 380, at least about 400, at least about 420, at least about 440, at least about 460, at least about 480, at least about 500 or at least about 550 mg.

E28. The method of any one of E1 to E27, wherein the anti-PD-1 antibody is administered at a flat dose ranging from at least about 200 mg to at least about 600 mg.

E29. The method of any one of E1 to E28, wherein the anti-PD-1 antibody is administered at a flat dose of about 240 mg, about 360 mg, about 480 mg, or about 560 mg.

E30. The method of any one of E1 to E29, wherein the anti-PD-1 antibody is administered at a flat dose of about 240 mg.

E31. The method of any one of E1 to E29, wherein the anti-PD-1 antibody is administered at a flat dose of about 360 mg.

E32. The method of any one of E1 to E31, wherein the anti-PD-1 antibody is administered once about every 1, 2, 3, or 4 weeks.

E33. The method of any one of E1 to E32, wherein the anti-PD-1 antibody is administered at a flat dose of about 240 mg, about 360 mg, about 480 mg, or about 560 mg about once every 2 weeks or every 3 weeks.

E34. The method of any one of E1 to E33, wherein the anti-PD-1 antibody is administered at a flat dose of about 240 mg about once every 2 weeks.

E35. The method of any one of E1 to E33, wherein the anti-PD-1 antibody is administered at a flat dose of about 360 mg about once every 3 weeks.

E36. The method of any one of E1 to E35, wherein the anti-PD-1 antibody is administered for as long as clinical benefit is observed or until unmanageable toxicity or disease progression occurs.

E37. The method of any one of E1 to E36, wherein the CD-122-biased agonist is administered at a dose ranging from at least about 0.0001 mg/kg to at least about 0.1 mg/kg body weight.

E38. The method of any one of E1 to E37, wherein the CD-122-biased agonist is administered at a dose ranging from at least about 0.001 mg/kg to at least about 0.01 mg/kg body weight.

E39. The method of any one of E1 to E38, wherein the CD-122-biased agonist is administered at a dose of about 0.003 mg/kg, about 0.004 mg/kg, about 0.005 mg/kg, about 0.006 mg/kg, about 0.007 mg/kg, about 0.008 mg/kg, about 0.009 mg/kg, or about 0.01 mg/kg body weight.

E40. The method of any one of E1 to E39, wherein the CD-122-biased agonist is administered at a dose of about 0.003 mg/kg body weight.

E41. The method of any one of E1 to E40, wherein the CD-122-biased agonist is administered at a dose of about 0.006 mg/kg body weight.

E42. The method of any one of E1 to E41, wherein the CD-122-biased agonist is administered once about every 1, 2, 3, or 4 weeks.

E43. The method of any one of E1 to E42, wherein the CD-122-biased agonist is administered at a dose of about 0.003 mg/kg body weight about every 2 weeks.

E44. The method of any one of E1 to E43, wherein the CD-122-biased agonist is administered at a dose of about 0.006 mg/kg body weight about every 2 weeks.

E45. The method of any one of E1 to E44, wherein the CD-122-biased agonist is administered at a dose of about 0.006 mg/kg body weight about every 3 weeks.

E46. The method of any of E1 to E45, wherein the anti-PD-1 antibody is administered at a dose of about 360 mg every 3 weeks and the CD-122-biased agonist is administered at a dose of about 0.006 mg/kg body weight about every 3 weeks.

E47. The method of any one of E1 to E46, wherein the anti-PD-1 antibody and the CD-122-biased agonist are formulated for intravenous administration.

E48. The method of any one of E1 to E47, wherein the anti-PD-1 antibody and the CD-122-biased agonist are administered sequentially.

E49. The method of any one of E1 to E48, wherein the anti-PD-1 antibody and the CD-122-biased agonist are administered within 30 minutes of each other.

E50. The method of any one of E1 to E49, wherein the anti-PD-1 antibody is administered before the CD-122-biased agonist.

E51. The method of any one of E1 to E50, wherein the CD-122-biased agonist is administered before the anti-PD-1 antibody.

E52. The method of any one of E1 to E51, wherein the anti-PD-1 antibody and the CD-122-biased agonist are administered concurrently in separate compositions.

E53. The method of any one of E1 to E51, wherein the anti-PD-1 antibody and the CD-122-biased agonist are admixed as a single composition for concurrent administration.

E54. The method of any one of E1 to E52, wherein the anti-PD-1 antibody is administered at a subtherapeutic dose.

E55. The method any one of E1 to E53, wherein the CD-122-biased agonist is administered at a subtherapeutic dose.

E56. The method any one of E1 to E55, wherein the anti-PD-1 antibody and the CD-122-biased agonist are each administered at a subtherapeutic dose.

E57. The method of any one of E1 to E56, wherein the tumor comprises one or more cells that express PD-L1, PD-L2, or both.

E58. The method of any one of E1 to E57, wherein the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration.

E59. The method of any one of E1 to E58, wherein the administration of the anti-PD-1 antibody and the CD-122-biased agonist reduces the size of the tumor relative to the size of the tumor prior to the administration.

E60. The method of E59, wherein the size of the tumor is reduced by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% as compared to the size of the tumor prior to the administration.

E61. The method of any one of E1 to E60, wherein the anti-PD-1 antibody is OPDIVO®.

E62. The method of any one of E1 to E61, wherein the subject received at least one prior chemotherapy treatment.

E63. A kit for treating a subject afflicted with a cancer, the kit comprising: (a) a dosage ranging from about 10 mg to about 600 mg of an anti-PD-1 antibody; (b) a dosage ranging from about 0.0001 mg to about 0.1 mg of a CD-122-biased agonist; (c) instructions for using the anti-PD-1 antibody and the CD-122-biased agonist in the method of any of E1 to E61.

### BRIEF DESCRIPTION OF THE DRAWINGS

FIG. 1A is a graphical representation of preclinical data showing the effects of various therapies on mean tumor size, including an anti-CTLA-4 antibody, an anti-PD-1 antibody, a CD-122-biased agonist, anti-CTLA-4+anti-PD-1 antibodies, a CD-122-biased agonist+anti-PD-1 antibody, and vehicle control. FIGS. 1B-1C are graphical representations of the level of PD-1⁺/CD8⁺ T cell proliferation in patient blood following treatment with CD-122-biased agonist monotherapy (FIG. 1B) and the fold change from baseline of CD8⁺ T cells and Treg cells present in tumor tissue following treatment with CD-122-biased agonist monotherapy (FIG. 1C).

FIG. 2 is a schematic of a Phase 1b dose escalation and expansion study.

FIG. 3 is a graphical representation illustrating the best percent change in target lesions by tumor type and dose for 36 subjects in a Phase 1b study. Patients were administered a combination therapy comprising a CD-122-biased agonist and nivolumab, as indicated. * Best overall response is PD (SD for target lesions, PD per non-target lesions); # Best overall response is SD (PR for target lesions, PD per new lesion at confirmatory scan); + Best overall response is PR (CR for target lesions, non-target lesions still present). Data are shown for patients with post-baseline scans that included assessment of target lesions. Two patients were not included in the figure: one patient was discontinued from the study due to clinical progression before the first post-baseline tumor assessment, and one patient on treatment did not have a post-baseline scan.

FIGS. 4A-4C are graphical representations of the percent change in target lesions over time (FIG. 4A), percent change from baseline in target lesions (FIG. 4B), and the time to and duration of response (FIG. 4C) for stage IV treatment-naive melanoma patients (n=11). Horizontal dotted lines indicate the thresholds for PD and response according to RECIST (version 1.1) criteria (FIGS. 4A-4B). # Best Overall Response is SD (PR for target lesions, PD per new lesion on confirmatory scan); + Best Overall response is PR (CR for target lesions, non-target lesions still present) (FIGS. 4A-4B). + Best Overall response is PR (CR for target lesions, non-target lesions still present) (FIG. 4C).

FIGS. 5A-5C are graphical representations of the percent change in target lesions over time (FIG. 5A), percent change from baseline in target lesions (FIG. 5B), and the time to and duration of response (FIG. 5C) for stage IV treatment-naïve first line renal cell carcinoma (RCC 1L) patients (n=13). Efficacy was evaluable for patients with more than 1 or more than 2 post baseline scans. Horizontal dotted lines indicate the thresholds for PD and response according to RECIST (version 1.1) criteria (FIGS. 5A-5B). # Best Overall Response is SD (PR for target lesions, PD per new lesion on confirmatory scan); + Best Overall response is PR (CR for target lesions, non-target lesions still present) (FIGS. 5A-5B).

FIGS. 6A-6C are graphical representations of the percent change in target lesions over time (FIG. 6A), percent change from baseline in target lesions (FIG. 6B), and the time to and duration of response (FIG. 6C) for stage IV treatment-naïve, PD-L1 negative non-small cell lung carcinoma (NSCLC) patients (first and second line; n=4). Horizontal dotted lines indicate the thresholds for PD and response according to RECIST (version 1.1) criteria (FIGS. 6A-6B). # Best Overall Response is SD (PR for target lesions, PD per new lesion on confirmatory scan); + Best Overall response is PR (CR for target lesions, non-target lesions still present) (FIGS. 6A-6B).

FIG. 7 is a graphical representation of the change in tumor size relative to baseline for 38 melanoma patients administered first line therapy comprising a combination of a CD-122-biased agonist and nivolumab. Per protocol, efficacy evaluable is defined as patients with ≥1 post baseline scan. 3 patients discontinued prior to 1st scan (due to TEAE [n=1] and Physicians Decision [n=2]). † 11 evaluable LDH >ULN. One patient not represented in the plot had target lesions per protocol by Investigator assessment but did not have target lesion at baseline by BICR. Patient achieved SD based on non-target lesion during the study. #: Best overall response is PD. *: Best overall response is SD. +: Best overall response is PR with −100% reduction of target lesions. §: Best overall response of CR is unconfirmed; PR confirmed.

FIG. 8 is a graphical representation of the change in tumor size relative to baseline for 38 melanoma patients plotted against the duration of treatment, following administration of a combination therapy of a CD-122-biased agonist and nivolumab. Per protocol, efficacy evaluable is defined as patients with ≥1 post baseline scan. Three patients discontinued prior to 1st scan (due to TEAE [n=1] and Physicians Decision [n=2]). Three responders progressed after six months of response. All three patients sustained tumor control of target lesions (−100%, −100%, −50%) with new lesions resulting in progression. One patient not represented in the plot had target lesions per protocol by Investigator assessment but did not have target lesion at baseline by BICR. Patient achieved stable disease (SD) based on non-target lesion during the study.

FIG. 9 is a schematic representation of the biomarker methodology for melanoma patients administered first line therapy comprising a combination of a CD-122-biased agonist and nivolumab. C=cycle; D=day (e.g., C1D1=cycle 1, day 1).

FIGS. 10A-10B are graphical representations of the level of CD-122-biased agonist active cytokine over-laid with the number of lymphocytes present in melanoma patients over time following administration of a first line therapy comprising a CD-122-biased agonist monotherapy (FIG. 10A) or a combination of a CD-122-biased agonist and nivolumab (FIG. 10B). Lymphocyte levels were obtained from standard hematology analysis (N=17 EXCEL and N=328 PIVOT-02). CD-122-biased agonist-AC (CD-122-biased agonist active cytokine, 2-PEG and 1-PEG IL-2) was measured by a qualified method (N=17 EXCEL and N=48 PIVOT-02).

FIGS. 11A-11B are graphical representations of the number of lymphocytes in the blood of melanoma patients following repeated administration of a first line comprising a CD-122-biased agonist monotherapy (FIG. 11A) or a combination of a CD-122-biased agonist and nivolumab (FIG. 11B). Lymphocyte levels were obtained from standard hematology analysis. All patients with data from the monotherapy trial EXCEL (N=17) and all first-line melanoma patients in the combination therapy enrolled in PIVOT-02 (N=41, Mean±SE) were included in the analysis.

FIGS. 12A-12B are graphical representations of the percent of antigen-experienced T cells (FIG. 12A; as represented by HLA DR+ cells) and the level of cell surface expression of ICOS on T cells (FIG. 12B) in cycle 1 relative to baseline for CD4+ T cells and CD8+ T cells. HLA-DR-positive T cells were enumerated using flow cytometry and presented as proportion (%) of each parent cell population. All patients with matched D1 and D8 Cycle 1 samples were included in the analysis (N=9; bars show median for each population) and fold-change from D8/D1 provided. * p<0.05 for D8 vs. D1. ICOS-positive T cells were enumerated using flow cytometry and cell surface expression of ICOS was calculated from a reference curve of Molecules of Equivalent Staining Fluorochrome (MESF). All patients with matched D1 and D8 Cycle 1 samples were included in the analysis (N=9, bars show median for each population) and fold-change from D8/D1 provided. * p<0.05 for D8 vs Dl.

FIGS. 13A-13B are images of immunofluorescence staining of tumor biopsies taken from a representative melanoma patient treated with a combination of a CD-122-biased agonist and nivolumab taken at baseline (FIG. 13A) and week 3 following treatment (FIG. 13B). Immunofluorescence staining was performed using Vectra with the indicated staining reagents. Images shown were obtained at 20× magnification. DAPI stains DNA, SOX-10 is a melanoma tumor antigen, CD3/CD8 stain T cells, CD68 stains macrophage. IHC for CD8 was obtained by standard methods. All first line melanoma patients with matched baseline and week 3 biopsy (N=8) were included in the analysis. FIG. 13C is a graphical representation of the change in CD8 infiltrate IHC staining in tumor biopsies taken from melanoma patients treated with a combination of a CD-122-biased agonist and nivolumab taken at baseline at baseline and week 3 following treatment. The representative patient used for FIGS. 13A-13B (“Patient A”) is indicated in FIG. 13C.

FIG. 14A is a volcano plot of differential expression on-treatment relative to pre-treatment. EdgeSeq was performed on all available samples, N=11 Baseline (BL) and N=5 Week 3 (W3). Only two patients had matching BL and W3 samples. Volcano Plot: points in Q1 and Q3 are both statistically significant (p-value≤0.05) and are over 2-fold higher (in linear space). Vertical dashed lines show 2-fold increase/decrease, horizontal dashed line shows threshold for statistical significance. FIGS. 14B-14E are bar graphs illustrating expression of genes encoding cell activation and co-inhibitory receptors (FIG. 14B; 4-1BB, CD86, PD-1, and LAG3), proteins with cytotoxic effector functions (FIG. 14C; perforin, granzyme, and IFNg), the melanoma tumor antigen SLC7A5 (FIG. 14D), and Th2/TH17 and inhibitory cytokines (FIG. 14E; IL17A, RORC, IL4, GATA3, and TGFB1) at week 3 relative to baseline. Stars indicate statistically significant genes (p-value≤0.05).

FIG. 15 is a graphical representation of the distribution of TCR clones at baseline and a week 3 for a select melanoma patient following first-line treatment with a combination of a CD-122-biased agonist and nivolumab. The percent of TIL at week 3 was found to be 4.4±1% (N=7). Tumor biopsy was processed to nucleic acid and used for TCR repertoire analysis using immunoSEQ. All first line melanoma patients (N=7) with matched baseline and week 3 samples are reported as % productive frequency. TCR Clones more abundant at Baseline are shown in red and clones more abundant at week 3 are shown in blue. Dark grey dots are not significant between time points and light gray dots are excluded for low abundance. The gray dashed line lists frequency equality and the red dashed line identifies the population used for statistical comparison. New T Cell infiltrates are shown in the oval and summarized for N=7 in the box above.

FIG. 16 is a graphical representation, illustrating the correlation between baseline CD8+ tumor infiltrating lymphocytes and PD-L1 expression on best overall response in melanoma patients following first line treatment with a combination therapy comprising a CD-122-biased agonist. Circles indicate complete response (CR), squares indicate partial response (PR), triangles indicate stable disease (SD), and asterisks indicate progressive disease (PD). Baseline tumor biopsies were evaluated by immunohistochemistry for CD8 cell counts (N=26), and PD-L1 expression (N=26) using the 28-8 method, or tumor mutation burden (TMB, N=12) using the Foundation TMB method. Each patient with matched baseline CD8 and % PD-L1 were plotted as x/y coordinates and correlated with BOR. Each symbol represents an individual patient (CR: N=7, PR: N=9, SD: N=4, and PD: N=6).

### DETAILED DESCRIPTION OF THE DISCLOSURE

This disclosure relates to methods for treating a tumor in a subject comprising administering to the subject an anti-Programmed Death-1 (PD-1) antibody and a CD-122-biased agonist. In some embodiments, the CD-122-biased agonist comprises an interleukin-2 protein conjugated to a polymer, such as a water-soluble polymer. In certain embodiments, the tumor is derived from a melanoma, a renal cell carcinoma (RCC), a non-small cell lung carcinoma (NSCLC), a urothelial cancer (UC), a breast cancer, or any combination thereof.

### Terms

In order that the present disclosure can be more readily understood, certain terms are first defined. As used in this application, except as otherwise expressly provided herein, each of the following terms shall have the meaning set forth below. Additional definitions are set forth throughout the application.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

“Administering” refers to the physical introduction of a therapeutic agent to a subject, using any of the various methods and delivery systems known to those skilled in the art. Exemplary routes of administration, e.g., for the anti-PD-1 antibody and/or the CD-122-biased agonist, include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises at least three constant domains, C_{H1}, C_{H2} and C_{H3}. Each light chain comprises a light chain variable region (abbreviated herein as V_{L}) and a light chain constant region. The light chain constant region comprises one constant domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

An immunoglobulin can derive from any of the commonly known isotypes, including but not limited to IgA, secretory IgA, IgG, and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or non-human antibodies; wholly synthetic antibodies; and single chain antibodies. A non-human antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

An “isolated antibody” refers to an antibody that is substantially free of other antibodies having different antigenic specificities (e.g., an isolated antibody that binds specifically to PD-1 is substantially free of antibodies that bind specifically to antigens other than PD-1). An isolated antibody that binds specifically to PD-1 can, however, have cross-reactivity to other antigens, such as PD-1 molecules from different species. Moreover, an isolated antibody can be substantially free of other cellular material and/or chemicals. In one embodiment, an antibody includes a conjugate attached to another agent (e.g., small molecule drug).

The term “monoclonal antibody” (mAb) refers to a non-naturally occurring preparation of antibody molecules of single molecular composition, i.e., antibody molecules whose primary sequences are essentially identical, and which exhibits a single binding specificity and affinity for a particular epitope. A monoclonal antibody is an example of an isolated antibody. Monoclonal antibodies can be produced by hybridoma, recombinant, transgenic, or other techniques known to those skilled in the art.

A “human antibody” (HuMAb) refers to an antibody having variable regions in which both the FRs and CDRs are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibodies” and “fully human antibodies” and are used synonymously.

A “humanized antibody” refers to an antibody in which some, most, or all of the amino acids outside the CDRs of a non-human antibody are replaced with corresponding amino acids derived from human immunoglobulins. In one embodiment of a humanized form of an antibody, some, most, or all of the amino acids outside the CDRs have been replaced with amino acids from human immunoglobulins, whereas some, most, or all amino acids within one or more CDRs are unchanged. Small additions, deletions, insertions, substitutions or modifications of amino acids are permissible as long as they do not abrogate the ability of the antibody to bind to a particular antigen. A “humanized antibody” retains an antigenic specificity similar to that of the original antibody. In some embodiments, the CDRs of a humanized antibody contain CDRs from a non-human, mammalian antibody. In other embodiments, the CDRs of a humanized antibody contain CDRs from an engineered, synthetic antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen antibody” refers to an antibody that binds specifically to the antigen. For example, an anti-PD-1 antibody binds specifically to PD-1.

An “antigen-binding portion” of an antibody (also called an “antigen-binding fragment”) refers to one or more fragments of an antibody that retain the ability to bind specifically to the antigen bound by the whole antibody.

A “cancer” refers a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor. For example, a “tumor derived from” a melanoma refers to a tumor that is the result of a metastasized melanoma. Because the distal tumor is derived from the pre-metastasis tumor, the “derived from” tumor can also comprise the pre-metastasis tumor, e.g., a tumor derived from a melanoma can comprise a melanoma.

“CD-122,” “interleukin-2 receptor β,” “IL-2Rβ,” or “IL2RB” refers to the beta subunit of a receptor for interleukin 2 (IL-2). CD-122 dimerizes with the IL-2R alpha subunit and further interacts with the IL-2R gamma subunit on the surface of immune cells to form an IL-2 receptor. Binding of IL-2 with the IL-2Rαβγ complex promotes proliferation of CD4⁺ Treg cells. Conversely, CD-122 also dimerizes with the γ subunit alone to form a IL-2Rβγ complex. Binding of IL-2 with the IL-2Rβγ complex drives proliferation of natural killer (NK) cells, CD8⁺ T cells, and CD4⁺ helper T cells. Accordingly, preferential activation of the IL-2Rβγ complex promotes an immune response, whereas activation of the IL-2Rαβγ complex promotes an immunosuppressive response.

A “CD-122 agonist,” “CD-122 biased agonist,” “IL-2Rβ-biased agonist,” or an “IL-2Rβ agonist” as used herein refers to any molecule capable of activating or stimulating CD-122 or IL-2Rβ. The agonist can comprise a small molecule, a polymer, a polypeptide, or any combination thereof. In some embodiments, the CD-122-biased agonist comprises an IL-2 protein or a fragment thereof conjugated to a polymer. In some embodiments, the CD-122-biased agonist binds and activates IL-2Rβγ over IL-2Rαβγ. In some embodiments, the CD-122-biased agonist selectively binds and activates IL-2Rβγ over IL-2Rαβγ. In certain embodiments, the CD-122-biased agonist does not bind IL-2Rαβγ. In particular embodiments, the CD-122-biased agonist comprises Formula I:

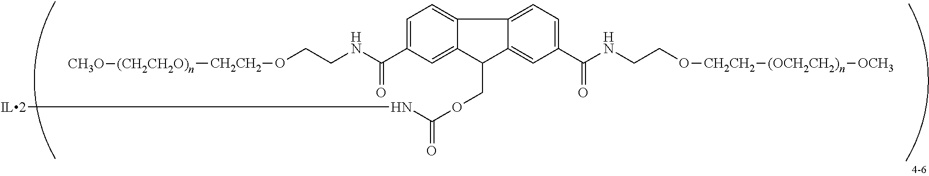

Formula (I), also referred to as (2,7-(bis-methoxyPEG-carboxyamide)(9H-fluorene-9-yl)methyl N-carbamate)₄₋₆interleukin-2.

A “polymer,” as used herein, refers to a non-peptidic molecule comprising multiple repeating subunits. The polymer can be naturally occurring or synthetic. In some embodiments, the polymer comprises a water-soluble polymer. In some embodiments, the polymer is a water-soluble polymer. In some embodiments, the polymer is a polyethylene glycol (PEG). In some embodiments, the polymer is comprised in the following formula (II):

A binding molecule, e.g., CD-122-biased agonist, “preferentially binds” to a receptor, e.g., IL-2Rβγ, if it binds with greater affinity, avidity, more readily, and/or with greater duration than it binds to other substances, e.g., IL-2Rαβγ. For example, a CD-122-biased agonist that preferentially binds to IL-2Rβγ is a molecule that binds IL-2Rβγ with greater affinity, avidity, more readily, and/or with greater duration than it binds to other IL-2 R, especially IL-2Rαβγ. For example, a CD-122-biased agonist preferentially binds to IL-2Rβγ if more than 50%, 60%, 70%, 80%, 90%, or 95% of the CD-122-biased agonist binds to IL-2Rβγ in the presence of both IL-2Rβγ and IL-2Rαβγ on the surface of cells. It is also understood by reading this definition that, for example, a CD-122-biased agonist (or moiety or epitope) that preferentially binds to a first target, e.g., IL-2Rβγ, may or may not preferentially bind to a second target, e.g., IL-2Rαβγ. As such, “preferential binding” does not necessarily require (although it can include) exclusive binding. Thus, in some aspects, “preferential binding” can be “exclusive binding.” To exemplify these concepts, if 50% of a CD-122-biased agonist specifically binds to IL-2Rβγ and 50% specifically binds to IL-2Rαβγ, such binding would be “non-selective” or “non-preferential.” If less than 50% of a CD-122-biased agonist binds to IL-2Rαβγ and more than 50% binds to IL-2Rβγ, the CD-122-biased agonist would “preferentially bind” to IL-2Rβγ. If the CD-122-biased agonist does not bind to IL-2Rαβγ and only binds to IL-2Rβγ, the CD-122-biased agonist would “exclusively bind” to IL-2Rβγ.

The term “immunotherapy” refers to the treatment of a subject afflicted with, at risk of contracting, or suffering a recurrence of a disease by a method comprising inducing, enhancing, suppressing, or otherwise modifying an immune response.

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease.

“Programmed Death-1” (PD-1) refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

“Programmed Death Ligand-1” (PD-L1) is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

A “subject” includes any human or non-human animal. The term “nonhuman animal” includes, but is not limited to, vertebrates such as nonhuman primates, sheep, dogs, and rodents such as mice, rats, and guinea pigs. In some embodiments, the subject is a human. The terms “subject” and “patient” are used interchangeably herein.

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, “subtherapeutic dose” means a dose of a therapeutic compound (e.g., an antibody and/or an agonist) that is lower than the usual or typical dose of the therapeutic compound when administered alone for the treatment of a hyperproliferative disease (e.g., cancer).

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

By way of example for the treatment of tumors, a therapeutically effective amount of an anti-cancer agent inhibits cell growth or tumor growth by at least about 10%, by at least about 20%, by at least about 30%, by at least about 40%, by at least about 50%, by at least about 60%, by at least about 70%, or by at least about 80%, by at least about 90%, at least about 95%, or at least about 100% relative to untreated subjects.

In other embodiments of the disclosure, tumor regression can be observed and continue for a period of at least about 20 days, at least about 30 days, at least about 40 days, at least about 50 days, or at least about 60 days. Notwithstanding these ultimate measurements of therapeutic effectiveness, evaluation of immunotherapeutic drugs must also make allowance for “immune-related response patterns.”

An “immune-related response pattern” refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents can require long-term monitoring of the effects of these agents on the target disease.

A therapeutically effective amount of a drug includes a “prophylactically effective amount,” which is any amount of the drug that, when administered alone or in combination with an anti-cancer agent to a subject at risk of developing a cancer (e.g., a subject having a pre-malignant condition) or of suffering a recurrence of cancer, inhibits the development or recurrence of the cancer. In some embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

The use of the term “flat dose” with regard to the methods and dosages of the disclosure means a dose that is administered to a patient without regard for the weight or body surface area (BSA) of the patient. The flat dose is therefore not provided as a mg/kg dose (i.e., a weight-based dosage), but rather as an absolute amount of the agent (e.g., the CD-122-biased agonist and/or anti-PD-1 antibody). For example, a 60 kg person and a 100 kg person would receive the same dose of an antibody (e.g., 360 mg of an anti-PD-1 antibody).

The term “weight-based dose,” as referred to herein, means that a dose administered to a patient is calculated based on the weight of the patient. For example, when a patient with 60 kg body weight requires 3 mg/kg of an anti-PD-1 antibody, one can calculate and use the appropriate amount of the anti-PD-1 antibody (i.e., 180 mg) for administration.

The use of the term “fixed dose” with regard to a method of the disclosure means that two or more different anti-cancer agents in a single composition (e.g., anti-PD-1 antibody and a CD-122-biased agonist) are present in the composition in particular (fixed) ratios with each other. In some embodiments, the fixed dose is based on the weight (e.g., mg) of the anti-cancer agents. In certain embodiments, the fixed dose is based on the concentration (e.g., mg/ml) of the anti-cancer agents.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The terms “once about every week,” “once about every two weeks,” or any other similar dosing interval terms as used herein mean approximate numbers. “Once about every week” can include every seven days±one day, i.e., every six days to every eight days. “Once about every two weeks” can include every fourteen days±three days, i.e., every eleven days to every seventeen days. Similar approximations apply, for example, to once about every three weeks, once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

Methods of the Disclosure

The present disclosure is directed to a method for treating a tumor or a subject afflicted with a tumor comprising administering to the subject an antibody that binds specifically to a Programmed Death-1 (PD-1) receptor and inhibits PD-1 activity (“anti-PD-1 antibody”) or an antibody or an antigen-binding portion thereof that binds specifically to a Programmed Death Ligandl (PD-L1) receptor and inhibits PD-L1 activity (“anti-PD-L1 antibody”) and a CD-122-biased agonist. In some embodiments, the CD-122-biased agonist comprises an IL-2 protein conjugated to a polymer. In some embodiments, the polymer comprises a water-soluble polymer. In some embodiments, the polymer is a water-soluble polymer.

In some embodiments, the tumor is derived from a melanoma, a renal cell carcinoma (RCC), a non-small cell lung carcinoma (NSCLC), a urothelial cancer (UC), a breast cancer, or any combination thereof. In some embodiments, the breast cancer is a triple negative breast cancer (TNBC). In certain embodiments, the administering treats the tumor.

In other embodiments, the presently described combination therapy can be used to treat a patient suffering from any condition that can be remedied or prevented by this method. Exemplary conditions are cancers, such as, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell cancer, basal cell cancer, adenocarcinoma, sweat gland cancer, sebaceous gland cancer, papillary cancer, papillary adenocarcinomas, cystadenocarcinoma, medullary cancer, bronchogenic cancer, renal cell cancer, hepatoma, bile duct cancer, choriocarcinoma, seminoma, embryonal cancer, Wilms' tumor, cervical cancer, testicular cancer, lung cancer, small cell lung cancer, bladder cancer, epithelial cancer, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma and leukemias.

The anti-PD-1 antibody or anti-PD-L1 antibody in combination with a CD-122-biased agonist is capable of driving new T cell clones into the tumor microenvironment, promoting new cell priming, promoting T cell trafficking, or any combination thereof.

In some embodiments, the CD-122-biased agonist interacts with an IL-2Rβγ on the surface of a cell. In certain embodiments, the CD-122-biased agonist preferentially interacts with an IL-2Rβγ on the surface of a cell in the presence of IL-2Rαβγ. In some embodiments, the CD-122-biased agonist interacts more strongly with an IL-2Rβγ on the surface of a cell than the CD-122-biased agonist interacts with an IL-2Rαβγ on the surface of the cell. In certain embodiments, the CD-122-biased agonist has a higher affinity for an IL-2Rβγ on the surface of a cell than an IL-2Rαβγ on the surface of the cell. In some embodiments, the affinity of the CD-122-biased agonist is at least about 1.5 fold, at least about 2 fold, at least about 2.5 fold, at least about 3 fold, at least about 3.5 fold, at least about 4 fold, at least about 4.5 fold, at least about 5 fold, at least about 6 fold, at least about 7 fold, at least about 8 fold, at least about 9 fold, or at least about 10 fold higher for an IL-2Rβγ on the surface of a cell than an IL-2Rαβγ on the surface of the cell. In some embodiments, the CD-122 interacts exclusively with an IL-2Rβγ on the surface of a cell. In certain embodiments, the CD-122 does not interact with an IL-2Rαβγ on the surface of a cell.

In some embodiments, the CD-122-biased agonist interacts with an IL-2Rβγ on the surface of a cell, wherein the cell is an immune cell. In some embodiments, the cell is selected from the group consisting of natural killer (NK) cell, a CD4⁺ cell, a CD8⁺ cell, and any combination thereof. In some embodiments, the CD-122-biased agonist promotes clonal expansion of NK cells. In some embodiments, the CD-122-biased agonist promotes clonal expansion of CD4⁺ helper T cells. In some embodiments, the CD-122-biased agonist promotes clonal expansion of CD8⁺ T cells. In some embodiments, binding of the CD-122-biased agonist with an IL-2Rβγ on the surface of the cell suppresses clonal expansion of CD4⁺ Treg cells. In some embodiments, the CD-122-biased agonist does not promote clonal expansion of CD4+ Treg cells. In some embodiments, the CD-122-biased agonist promotes an anti-tumor immune response by increasing the number of NK cells, CD4⁺ helper T cells, and/or CD8⁺ cells. In some embodiments, the CD-122-biased agonist promotes an anti-tumor immune response by suppressing an immunosuppressive response by suppressing the expansion of CD4⁺ Treg cells.

In certain embodiments, the administration of the CD-122-biased agonist increases proliferation of tumor infiltrating lymphocytes (TILs) in the tumor as compared to the proliferation of TILs in the tumor prior to the administration. In some embodiments, the administration of the CD-122-biased agonist increases proliferation of TILs in the tumor by at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 400%, at least about 500%, at least about 600%, at least about 700%, at least about 800%, at least about 900%, or at least about 1000% as compared to the proliferation of TILs in the tumor prior to the administration. In certain embodiments, the administration of the CD-122-biased agonist increases the number of tumor infiltrating lymphocytes (TILs) in the tumor as compared to the number of TILs in the tumor prior to the administration. In some embodiments, the administration of the CD-122-biased agonist increases the number of TILs in the tumor by at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 400%, at least about 500%, at least about 600%, at least about 700%, at least about 800%, at least about 900%, or at least about 1000% as compared to the number of TILs in the tumor prior to the administration.

In some embodiments, the administration of the CD-122-biased agonist increases PD-1 expression on effector T cells in the subject as compared to the PD-1 expression on effector T cells prior to the administration. In certain embodiments, the administration of the CD-122-biased agonist increases PD-1 expression on effector T cells in the subject by at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 400%, at least about 500%, at least about 600%, at least about 700%, at least about 800%, at least about 900%, or at least about 1000% as compared to the PD-1 expression on effector T cells prior to the administration.

In certain embodiments, the subject has received one, two, three, four, five or more prior cancer treatments. In other embodiments, the subject is treatment-naïve. In some embodiments, the subject has progressed on other cancer treatments. In certain embodiments, the prior cancer treatment comprised an immunotherapy. In other embodiments, the prior cancer treatment comprised a chemotherapy. In some embodiments, the tumor has reoccurred. In some embodiments, the tumor is metastatic. In other embodiments, the tumor is not metastatic.

In some embodiments, the subject has received a prior therapy to treat the tumor and the tumor is relapsed or refractory. In some embodiments, the subject has received a prior immuno-oncology (I-O) therapy to treat the tumor and the tumor is relapsed or refractory. In some embodiments, the subject has received more than one prior therapy to treat the tumor and the subject is relapsed or refractory. In other embodiments, the subject has received either an anti-PD-1 or anti-PD-L1 antibody monotherapy or a CD-122-biased agonist monotherapy.

In some embodiments, the previous line of therapy comprises a chemotherapy. In some embodiments, the chemotherapy comprises a platinum-based therapy. In some embodiments, the platinum-based therapy comprises a platinum-based antineoplastic selected from the group consisting of cisplatin, carboplatin, oxaliplatin, nedaplatin, triplatin tetranitrate, phenanthriplatin, picoplatin, satraplatin, and any combination thereof. In certain embodiments, the platinum-based therapy comprises cisplatin. In one particular embodiment, the platinum-based therapy comprises carboplatin.

In certain embodiments, the therapy of the present disclosure (e.g., administration of an anti-PD-1 antibody and a CD-122-biased agonist) effectively increases the duration of survival of the subject. For example, the duration of survival of the subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year or more when compared to another subject treated with only either another therapy or, only one of the two members of the combination therapy alone (e.g., an anti-PD-1 antibody alone) or an alternative combination therapy. In still other embodiments, the combination therapy of an anti-PD-1 antibody (e.g., nivolumab or pembrolizumab) and a CD-122-biased agonist increases the duration of survival of the subject at a level higher than (about one month higher than, about two months higher than, about three months higher than, about four months higher than, about five months higher than, about six months higher than, about seven months higher than, about eight months higher than, about nine months higher than, about ten months higher than, about eleven months higher than, or about one year higher than the duration of survival of the subject using a combination therapy of an anti-PD-L1 antibody (e.g., MPDL3280A or atezolizumab) and a CD-122-biased agonist.

In certain embodiments, the therapy of the present disclosure effectively increases the duration of progression-free survival of the subject. For example, the progression free survival of the subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year when compared to another subject treated with only either another therapy or only one of the two members of the combination therapy alone (e.g., an anti-PD-1 antibody alone or a CD-122-biased agonist alone) or an alternative combination therapy.

In certain embodiments, the therapy of the present disclosure effectively increases the response rate in a group of subjects. For example, the response rate in a group of subjects is increased by at least about 2%, at least about 3%, at least about 4%, at least about 5%, at least about 10%, at least about 15%, at least about 20%, at least about 25%, at least about 30%, at last about 35%, at least about 40%, at least about 45%, at least about 50%, at least about 55%, at least about 60%, at least about 70%, at least about 75%, at least about 80%, at least about 85%, at least about 90%, at least about 95%, at least about 99% or at least about 100% when compared to another group of subjects treated with only either another therapy or, only one of the two members of the combination therapy alone (e.g., an anti-PD-1 antibody alone or a CD-122-biased agonist alone) or an alternative combination therapy.

Anti-PD-1 Antibodies Useful for the Disclosure

Anti-PD-1 antibodies that are known in the art can be used in the presently described methods, and those provided herein are non-limiting examples. Various human monoclonal antibodies that bind specifically to PD-1 with high affinity have been disclosed in U.S. Pat. No. 8,008,449. Anti-PD-1 human antibodies disclosed in U.S. Pat. No. 8,008,449 have been demonstrated to exhibit one or more of the following characteristics: (a) bind to human PD-1 with a K_{D} of 1×10⁻⁷ M or less, as determined by surface plasmon resonance using a Biacore biosensor system; (b) do not substantially bind to human CD28, CTLA-4 or ICOS; (c) increase T-cell proliferation in a Mixed Lymphocyte Reaction (MLR) assay; (d) increase interferon-γ production in an MLR assay; (e) increase IL-2 secretion in an MLR assay; (f) bind to human PD-1 and cynomolgus monkey PD-1; (g) inhibit the binding of PD-L1 and/or PD-L2 to PD-1; (h) stimulate antigen-specific memory responses; (i) stimulate antibody responses; and (j) inhibit tumor cell growth in vivo. Anti-PD-1 antibodies usable in the present disclosure include monoclonal antibodies that bind specifically to human PD-1 and exhibit at least one, in some embodiments, at least five, of the preceding characteristics.

Other anti-PD-1 monoclonal antibodies have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540 each of which is incorporated by reference in its entirety.

In some embodiments, the anti-PD-1 antibody is selected from the group consisting of nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), spartalizumab (Novartis, also known as PDR001; see WO 2015/112900), MEDI-0680 (AstraZeneca, also known as AMP-514; see WO 2012/145493), cemiplimab (Regeneron, also known as REGN-2810; see WO 2015/112800), JS001 (TAIZHOU JUNSHI PHARMA; see Si-Yang Liu et al., J. Hematol. Oncol. 10:136 (2017)), tislelizumab (Beigene, also known as BGB-A317; see WO 2015/35606 and US 2015/0079109), INCSHR1210 (Jiangsu Hengrui Medicine, also known as SHR-1210; see WO 2015/085847; Si-Yang Liu et al., J. Hematol. Oncol. 10:136 (2017)), TSR-042 (Tesaro Biopharmaceutical, also known as ANB011; see WO2014/179664), GLS-010 (Wuxi/Harbin Gloria Pharmaceuticals, also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics; see WO 2017/19846), IBI308 (Innovent; see WO 2017/024465, WO 2017/025016, WO 2017/132825, and WO 2017/133540), and BCD-100 (Biocad).

In one embodiment, the anti-PD-1 antibody is nivolumab. Nivolumab is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56).

In another embodiment, the anti-PD-1 antibody is pembrolizumab. Pembrolizumab is a humanized monoclonal IgG4 (S228P) antibody directed against human cell surface receptor PD-1 (programmed death-1 or programmed cell death-1). Pembrolizumab is described, for example, in U.S. Pat. Nos. 8,354,509 and 8,900,587.

Anti-PD-1 antibodies usable in the disclosed methods also include isolated antibodies that bind specifically to human PD-1 and cross-compete for binding to human PD-1 with any anti-PD-1 antibody disclosed herein, e.g., nivolumab (see, e.g., U.S. Pat. Nos. 8,008,449 and 8,779,105; WO 2013/173223). In some embodiments, the anti-PD-1 antibody binds the same epitope as any of the anti-PD-1 antibodies described herein, e.g., nivolumab. The ability of antibodies to cross-compete for binding to an antigen indicates that these monoclonal antibodies bind to the same epitope region of the antigen and sterically hinder the binding of other cross-competing antibodies to that particular epitope region. These cross-competing antibodies are expected to have functional properties very similar those of the reference antibody, e.g., nivolumab, by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g., WO 2013/173223).

In certain embodiments, the antibodies that cross-compete for binding to human PD-1 with, or bind to the same epitope region of human PD-1 antibody, nivolumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-1 antibodies usable in the methods of the disclosure also include antigen-binding portions of the above antibodies. It has been amply demonstrated that the antigen-binding function of an antibody can be performed by fragments of a full-length antibody.

Anti-PD-1 antibodies suitable for use in the disclosed methods or compositions are antibodies that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole antibodies in inhibiting ligand binding and up-regulating the immune system. In certain embodiments, the anti-PD-1 antibody or antigen-binding portion thereof cross-competes with nivolumab for binding to human PD-1.

In some embodiments, the anti-PD-1 antibody is administered at a dose ranging from 0.1 mg/kg to 20.0 mg/kg body weight once every 2, 3, 4, 5, 6, 7, or 8 weeks, e.g., 0.1 mg/kg to 10.0 mg/kg body weight once every 2, 3, or 4 weeks. In other embodiments, the anti-PD-1 antibody is administered at a dose of about 2 mg/kg, about 3 mg/kg, about 4 mg/kg, about 5 mg/kg, about 6 mg/kg, about 7 mg/kg, about 8 mg/kg, about 9 mg/kg, or 10 mg/kg body weight once every 2 weeks. In other embodiments, the anti-PD-1 antibody is administered at a dose of about 2 mg/kg, about 3 mg/kg, about 4 mg/kg, about 5 mg/kg, about 6 mg/kg, about 7 mg/kg, about 8 mg/kg, about 9 mg/kg, or 10 mg/kg body weight once every 3 weeks. In one embodiment, the anti-PD-1 antibody is administered at a dose of about 5 mg/kg body weight about once every 3 weeks. In another embodiment, the anti-PD-1 antibody, e.g., nivolumab, is administered at a dose of about 3 mg/kg body weight about once every 2 weeks. In other embodiments, the anti-PD-1 antibody, e.g., pembrolizumab, is administered at a dose of about 2 mg/kg body weight about once every 3 weeks.

The anti-PD-1 antibody useful for the present disclosure can be administered as a flat dose. In some embodiments, the anti-PD-1 antibody is administered at a flat dose of from about 100 to about 1000 mg, from about 100 mg to about 900 mg, from about 100 mg to about 800 mg, from about 100 mg to about 700 mg, from about 100 mg to about 600 mg, from about 100 mg to about 500 mg, from about 200 mg to about 1000 mg, from about 200 mg to about 900 mg, from about 200 mg to about 800 mg, from about 200 mg to about 700 mg, from about 200 mg to about 600 mg, from about 200 mg to about 500 mg, from about 200 mg to about 480 mg, or from about 240 mg to about 480 mg, In one embodiment, the anti-PD-1 antibody is administered as a flat dose of at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, at least about 520 mg, at least about 540 mg, at least about 550 mg, at least about 560 mg, at least about 580 mg, at least about 600 mg, at least about 620 mg, at least about 640 mg, at least about 660 mg, at least about 680 mg, at least about 700 mg, or at least about 720 mg at a dosing interval of about 1, 2, 3, 4, 5, 6, 7, 8, 9, or 10 weeks. In another embodiments, the anti-PD-1 antibody is administered as a flat dose of about 200 mg to about 800 mg, about 200 mg to about 700 mg, about 200 mg to about 600 mg, about 200 mg to about 500 mg, at a dosing interval of about 1, 2, 3, or 4 weeks.

In some embodiments, the anti-PD-1 antibody is administered as a flat dose of about 200 mg at about once every 3 weeks. In other embodiments, the anti-PD-1 antibody is administered as a flat dose of about 200 mg at about once every 2 weeks. In other embodiments, the anti-PD-1 antibody is administered as a flat dose of about 240 mg at about once every 2 weeks. In other embodiments, the anti-PD-1 antibody is administered as a flat dose of about 360 mg at about once every 3 weeks. In certain embodiments, the anti-PD-1 antibody is administered as a flat dose of about 480 mg at about once every 4 weeks.

Anti-PD-L1 Antibodies Useful for the Disclosure

Because anti-PD-1 and anti-PD-L1 target the same signaling pathway and have been shown in clinical trials to exhibit similar levels of efficacy in a variety of cancers, including renal cell carcinoma (see Brahmer et al. (2012) N Engl J Med 366:2455-65; Topalian et al. (2012a) N Engl J Med 366:2443-54; WO 2013/173223), an anti-PD-L1 antibody may be substituted for the anti-PD-1 antibody in any of the therapeutic methods disclosed herein. Anti-PD-L1 antibodies that are known in the art can be used in the methods of the present disclosure. Examples of anti-PD-L1 antibodies useful in the methods of the present disclosure include the antibodies disclosed in U.S. Pat. No. 9,580,507. Anti-PD-L1 human monoclonal antibodies disclosed in U.S. Pat. No. 9,580,507 have been demonstrated to exhibit one or more of the following characteristics: (a) bind to human PD-L1 with a K_{D} of 1×10⁻⁷ M or less, as determined by surface plasmon resonance using a Biacore biosensor system; (b) increase T-cell proliferation in a Mixed Lymphocyte Reaction (MLR) assay; (c) increase interferon-γ production in an MLR assay; (d) increase IL-2 secretion in an MLR assay; (e) stimulate antibody responses; and (f) reverse the effect of T regulatory cells on T cell effector cells and/or dendritic cells. Anti-PD-L1 antibodies usable in the present disclosure include monoclonal antibodies that bind specifically to human PD-L1 and exhibit at least one, in some embodiments, at least five, of the preceding characteristics.

In certain embodiments, the anti-PD-L1 antibody is selected from the group consisting of BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 31(suppl):3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), and CK-301 (Checkpoint Therapeutics; see Gorelik et al., AACR: Abstract 4606 (April 2016)).

In certain embodiments, the PD-L1 antibody is atezolizumab (TECENTRIQ®). Atezolizumab is a fully humanized IgG1 monoclonal anti-PD-L1 antibody.

In certain embodiments, the PD-L1 antibody is durvalumab (IMFINZI™). Durvalumab is a human IgG1 kappa monoclonal anti-PD-L1 antibody.

In certain embodiments, the PD-L1 antibody is avelumab (BAVENCIO®). Avelumab is a human IgG1 lambda monoclonal anti-PD-L1 antibody.

In other embodiments, the anti-PD-L1 monoclonal antibody is selected from the group consisting of 28-8, 28-1, 28-12, 29-8, 5H1, and any combination thereof.

Anti-PD-L1 antibodies usable in the disclosed methods also include isolated antibodies that bind specifically to human PD-L1 and cross-compete for binding to human PD-L1 with any anti-PD-L1 antibody disclosed herein, e.g., atezolizumab, durvalumab, and/or avelumab. In some embodiments, the anti-PD-L1 antibody binds the same epitope as any of the anti-PD-L1 antibodies described herein, e.g., atezolizumab, durvalumab, and/or avelumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., atezolizumab and/or avelumab, by virtue of their binding to the same epitope region of PD-L1. Cross-competing antibodies can be readily identified based on their ability to cross-compete with atezolizumab and/or avelumab in standard PD-L1 binding assays such as Biacore analysis, ELISA assays or flow cytometry (see, e.g., WO 2013/173223).

In certain embodiments, the antibodies that cross-compete for binding to human PD-L1 with, or bind to the same epitope region of human PD-L1 antibody as, atezolizumab, durvalumab, and/or avelumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-L1 antibodies usable in the methods of the disclosed disclosure also include antigen-binding portions of the above antibodies. It has been amply demonstrated that the antigen-binding function of an antibody can be performed by fragments of a full-length antibody.

Anti-PD-L1 antibodies suitable for use in the disclosed methods or compositions are antibodies that bind to PD-L1 with high specificity and affinity, block the binding of PD-1, and inhibit the immunosuppressive effect of the PD-1 signaling pathway. In any of the compositions or methods disclosed herein, an anti-PD-L1 “antibody” includes an antigen-binding portion or fragment that binds to PD-L1 and exhibits the functional properties similar to those of whole antibodies in inhibiting receptor binding and up-regulating the immune system. In certain embodiments, the anti-PD-L1 antibody or antigen-binding portion thereof cross-competes with atezolizumab, durvalumab, and/or avelumab for binding to human PD-L1.

The anti-PD-L1 antibody useful for the present disclosure can be any PD-L1 antibody that specifically binds to PD-L1, e.g., antibodies that cross-compete with durvalumab, avelumab, or atezolizumab for binding to human PD-1, e.g., an antibody that binds to the same epitope as durvalumab, avelumab, or atezolizumab. In a particular embodiment, the anti-PD-L1 antibody is durvalumab. In other embodiments, the anti-PD-L1 antibody is avelumab. In some embodiments, the anti-PD-L1 antibody is atezolizumab.

In some embodiments, the anti-PD-L1 antibody is administered at a dose ranging from about 0.1 mg/kg to about 20.0 mg/kg body weight, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg, about once every 2, 3, 4, 5, 6, 7, or 8 weeks.

In some embodiments, the anti-PD-L1 antibody is administered at a dose of about 15 mg/kg body weight at about once every 3 weeks. In other embodiments, the anti-PD-L1 antibody is administered at a dose of about 10 mg/kg body weight at about once every 2 weeks.

In other embodiments, the anti-PD-L1 antibody useful for the present disclosure is a flat dose. In some embodiments, the anti-PD-L1 antibody is administered as a flat dose of from about 200 mg to about 1600 mg, about 200 mg to about 1500 mg, about 200 mg to about 1400 mg, about 200 mg to about 1300 mg, about 200 mg to about 1200 mg, about 200 mg to about 1100 mg, about 200 mg to about 1000 mg, about 200 mg to about 900 mg, about 200 mg to about 800 mg, about 200 mg to about 700 mg, about 200 mg to about 600 mg, about 700 mg to about 1300 mg, about 800 mg to about 1200 mg, about 700 mg to about 900 mg, or about 1100 mg to about 1300 mg. In some embodiments, the anti-PD-L1 antibody is administered as a flat dose of at least about 240 mg, at least about 300 mg, at least about 320 mg, at least about 400 mg, at least about 480 mg, at least about 500 mg, at least about 560 mg, at least about 600 mg, at least about 640 mg, at least about 700 mg, at least 720 mg, at least about 800 mg, at least about 880 mg, at least about 900 mg, at least 960 mg, at least about 1000 mg, at least about 1040 mg, at least about 1100 mg, at least about 1120 mg, at least about 1200 mg, at least about 1280 mg, at least about 1300 mg, at least about 1360 mg, or at least about 1400 mg, at a dosing interval of about 1, 2, 3, or 4 weeks. In some embodiments, the anti-PD-L1 antibody is administered at a flat dose of about 1000 mg. In some embodiments, the anti-PD-L1 antibody is administered at a flat dose of about 1100 mg. In some embodiments, the anti-PD-L1 antibody is administered at a flat dose of about 1200 mg. In some embodiments, the anti-PD-L1 antibody is administered at a flat dose of about 1300 mg. In some embodiments, the anti-PD-L1 antibody is administered at a flat dose of about 1400 mg. In some embodiments, the anti-PD-L1 antibody is administered at a flat dose of about 1500 mg. In some embodiments, the anti-PD-L1 antibody is administered as a flat dose of about 1200 mg at about once every 3 weeks. In other embodiments, the anti-PD-L1 antibody is administered as a flat dose of about 800 mg at about once every 2 weeks.

CD-122-Biased Agonists

As used herein, a CD-122-biased agonist is a molecule that is capable of stimulating IL-2Rβ, and in particular the IL-2Rβγ complex. In certain embodiments, the CD-122-biased agonist comprises an IL-2 protein or a fragment thereof conjugated to a polymer, e.g., a water-soluble polymer, e.g., a polyethylene glycol (PEG) polymer. In some embodiments, the polymer comprises a PEG. In other embodiments, the PEG polymer is a branched polymer.

In other embodiments, the polymer is a water-soluble polymer, i.e., a non-peptidic, water soluble polymer. “Water-soluble, non-peptidic polymer” refers to a polymer that is at least 35% (by weight) soluble, greater than 70% (by weight), or greater than 95% (by weight) soluble, in water at room temperature. Typically, an unfiltered aqueous preparation of a “water-soluble” polymer transmits at least 75%, at least 80%, at least 90%, or at least 95%, of the amount of light transmitted by the same solution after filtering. In some embodiments, the water-soluble polymer is at least 95% (by weight) soluble in water or completely soluble in water. With respect to being “non-peptidic,” a polymer is non-peptidic when it has less than 35% (by weight) of amino acid residues.

“PEG” or “polyethylene glycol,” as used herein, is meant to encompass any water-soluble poly(ethylene oxide). Unless otherwise indicated, a “PEG polymer” or a polyethylene glycol is one in which substantially all (preferably all) monomeric subunits are ethylene oxide subunits, though, the polymer can contain distinct end capping moieties or functional groups, e.g., for conjugation. PEG polymers for use in the present invention will comprise one of the two following structures: “—(CH₂CH₂O)ₙ₋ₙ or “—(CH₂CH₂O)ₙ₋₁CH₂CH₂—,” depending upon whether or not the terminal oxygen(s) has been displaced, e.g., during a synthetic transformation. As stated above, for the PEG polymers, the variable (n) ranges from about 3 to 4000, and the terminal groups and architecture of the overall PEG can vary. “Branched”, in reference to the geometry or overall structure of a polymer, refers to a polymer having two or more polymer “arms” extending from a branch point or from a central moiety.

Typically, the weight-average molecular weight of the water-soluble polymer in the conjugate is from about 100 Daltons to about 150,000 Daltons. Exemplary ranges, however, include weight-average molecular weights in the range of greater than 5,000 Daltons to about 100,000 Daltons, in the range of from about 6,000 Daltons to about 90,000 Daltons, in the range of from about 10,000 Daltons to about 85,000 Daltons, in the range of greater than 10,000 Daltons to about 85,000 Daltons, in the range of from about 20,000 Daltons to about 85,000 Daltons, in the range of from about 53,000 Daltons to about 85,000 Daltons, in the range of from about 25,000 Daltons to about 120,000 Daltons, in the range of from about 29,000 Daltons to about 120,000 Daltons, in the range of from about 35,000 Daltons to about 120,000 Daltons, and in the range of from about 40,000 Daltons to about 120,000 Daltons.

Exemplary weight-average molecular weights for the water-soluble polymer include about 100 Daltons, about 200 Daltons, about 300 Daltons, about 400 Daltons, about 500 Daltons, about 600 Daltons, about 700 Daltons, about 750 Daltons, about 800 Daltons, about 900 Daltons, about 1,000 Daltons, about 1,500 Daltons, about 2,000 Daltons, about 2,200 Daltons, about 2,500 Daltons, about 3,000 Daltons, about 4,000 Daltons, about 4,400 Daltons, about 4,500 Daltons, about 5,000 Daltons, about 5,500 Daltons, about 6,000 Daltons, about 7,000 Daltons, about 7,500 Daltons, about 8,000 Daltons, about 9,000 Daltons, about 10,000 Daltons, about 11,000 Daltons, about 12,000 Daltons, about 13,000 Daltons, about 14,000 Daltons, about 15,000 Daltons, about 20,000 Daltons, about 22,500 Daltons, about 25,000 Daltons, about 30,000 Daltons, about 35,000 Daltons, about 40,000 Daltons, about 45,000 Daltons, about 50,000 Daltons, about 55,000 Daltons, about 60,000 Daltons, about 65,000 Daltons, about 70,000 Daltons, and about 75,000 Daltons. Branched versions of the water-soluble polymer (e.g., a branched 20,000 Dalton water-soluble polymer comprised of two 10,000 Dalton polymer chains) having a total molecular weight of any of the foregoing can also be used. In some embodiments, the weight-average of each branched PEG molecule is about 20,000 Daltons.

Molecular weight in the context of a water-soluble polymer, such as PEG, can be expressed as either a number average molecular weight or a weight average molecular weight. Unless otherwise indicated, all references to molecular weight herein refer to the weight average molecular weight. Both molecular weight determinations, number average and weight average, can be measured using gel permeation chromatography or other liquid chromatography techniques. Other methods for measuring molecular weight values can also be used, such as the use of end-group analysis or the measurement of colligative properties (e.g., freezing-point depression, boiling-point elevation, or osmotic pressure) to determine number average molecular weight or the use of light scattering techniques, ultracentrifugation or viscometry to determine weight average molecular weight. The polymers described herein are typically polydisperse (i.e., number average molecular weight and weight average molecular weight of the polymers are not equal), possessing low polydispersity values of preferably less than about 1.2, more preferably less than about 1.15, still more preferably less than about 1.10, yet still more preferably less than about 1.05, and most preferably less than about 1.03.

In some embodiments, the polymer portion is comprised in the following formula (II), including the carbamate linkage to an amino group of the interleukin-2 moiety (“IL-2”), where the “NH”˜ portion of the carbamate linkage represents an amino group of the interleukin-2 moiety:

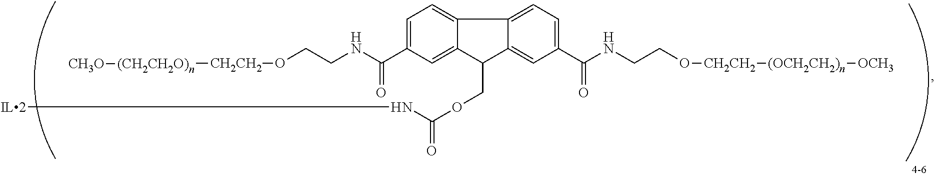

where (n) (outside the parentheses) has an average value of about 6, also referred to as (2,7-(bis-methoxyPEG-carboxyamide)(9H-fluorene-9-yl)methyl N-carbamate)_{6avg}interleukin-2. In some embodiments, the CD-122-biased agonist comprises conjugates according to the following formula (I):

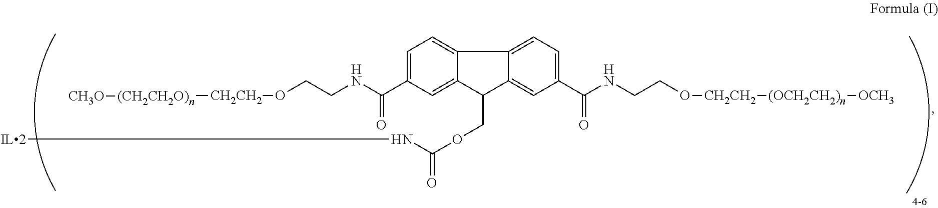

including its pharmaceutically acceptable salts, and each “n” is independently an integer from about 3 to about 1000. Representative ranges for each “n” include, for example, an integer from about 40 to about 550, or an integer from about 60 to about 500, or an integer from about 113 to about 400, or from 200-300. In certain embodiments, “n” in each of the polyethylene glycol chains is about 227 (i.e., where each polyethylene glycol chain extending from the central fluorenyl core has a weight average molecular weight of about 10,000 daltons, such that the weight average molecular weight of the overall branched PEG moiety is about 20,000 daltons), i.e., referred to herein as multi(2,7-(bis-methoxyPEG_{10kD}-carboxyamide)(9H-fluorene-9-yl)methyl N-carbamate)interleukin-2 or as (2,7-(bis-methoxyPEG_{10kD}-carboxyamide)(9H-fluorene-9-yl)methyl N-carbamate)₄₋₆interleukin-2. In some embodiments, the average degree of PEGylation for the composition is about 6 PEG molecules per interleukin-2 moiety. To determine an average degree of PEGylation for a polymer conjugate composition such as described in Formula (I), typically the protein is quantified by a method such as an bicinchoninic acid (BCA) assay or by UV analysis, to determine moles of protein in the sample. The PEG moieties are then released by exposing the sample to conditions in which the PEG moieties are released, and the released PEG is then quantified (e.g., by BCA or UV) and correlated with moles protein to determine average degree of PEGylation.

In one or more embodiments, the CD-122-biased agonist composition contains no more than 10% (based on a molar amount), preferably no more than 5% (based on a molar amount), of compounds encompassed by the following formula:

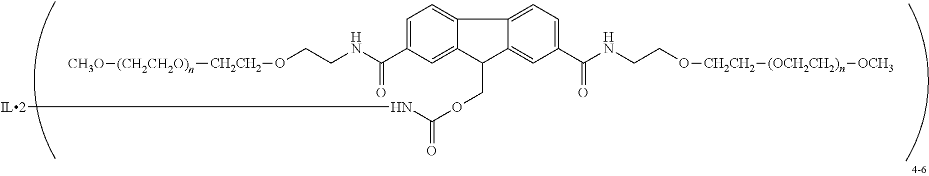

wherein IL-2 is an interleukin-2, and (n) (outside the parentheses) is an integer selected from the group consisting of 1, 2, 3, 7 and >7, and pharmaceutically acceptable salts thereof.

In some embodiments, the polymer is conjugated to an IL-2. In some embodiments, the IL2 is a recombinant IL-2. In some embodiments, the IL-2 is PROLEUKIN® (i.e., aldesleukin).

In some embodiments, the CD-122-biased agonist is biased for IL-2Rβγ over IL-2Rαβγ. In some embodiments, the CD-122-biased agonist binds IL-2Rβγ with greater affinity than IL-2Rαβγ. In some embodiments, the CD-122-biased agonist binds IL-2Rβγ with an affinity at least about 2-fold, at least about 3-fold, at least about 4-fold, at least about 5-fold, at least about 7-fold, at least about 10-fold, at least about 15-fold, at least about 20-fold, at least about 25-fold, or at least about 50-fold greater than the affinity of the CD-122-biased agonist for IL-2Rαβγ. In certain embodiments, the CD-122-biased agonist binds IL-2Rβγ with at least about 5-fold greater affinity than IL-2Rαβγ. In certain embodiments, the CD-122-biased agonist binds IL-2Rβγ with at least about 10-fold greater affinity than IL-2Rαβγ. In certain embodiments, the CD-122-biased agonist does not bind IL-2Rαβγ. In certain embodiments, the CD-122-biased agonist binds but does not activate or stimulate IL-2Rαβγ.

In some embodiments, the CD-122-biased agonist is long-acting. Non-limiting examples of long acting, IL-2RP-selective agonists are described in WO 2012/065086 and WO 2015/125159. In certain embodiments, the CD-122-biased agonist has an in vivo half-life that is greater than the in vivo half-life of IL-2. In some embodiments, the in vivo half-life of the CD-122-biased agonist is at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, or at least about 50-fold greater than the in vivo half-life of IL-2. In certain embodiments, the CD-122-biased agonist has an in vivo half-life that is at least about 5-fold greater than the in vivo half-life of IL-2. In certain embodiments, the CD-122-biased agonist has an in vivo half-life that is at least about 10-fold greater than the in vivo half-life of IL-2.

Dosing

In certain embodiments, the present methods comprise administering an effective amount of an anti-PD-1 antibody and an effective amount of a CD-122-biased agonist. An effective amount of an anti-PD-1 antibody and/or a CD-122-biased agonist can be a flat dose, a weight based dose, or both. Dosage regimens are adjusted to provide the optimum desired response, e.g., a maximal therapeutic response and/or minimal adverse effects.

In some embodiments, the anti-PD-1 antibody (e.g., nivolumab) is administered at a flat dose. In some embodiments, the anti-PD-1 antibody is administered at a flat dose ranging from at least about 200 mg to at least about 600 mg. In some embodiments, the anti-PD-1 antibody is administered at a flat dose of at least about 200 mg, at least about 210 mg, at least about 220 mg, at least about 230 mg, at least about 240 mg, at least about 250 mg, at least about 260 mg, at least about 270 mg, at least about 280 mg, at least about 290 mg, at least about 300 mg, at least about 310 mg, at least about 320 mg, at least about 330 mg, at least about 340 mg, at least about 350 mg, at least about 360 mg, at least about 370 mg, at least about 380 mg, at least about 390 mg, at least about 400 mg, at least about 410 mg, at least about 420 mg, at least about 430 mg, at least about 440 mg, at least about 450 mg, at least about 460 mg, at least about 470 mg, at least about 480 mg, at least about 490 mg, at least about 500 mg, at least about 510 mg, at least about 520 mg, at least about 530 mg, at least about 540 mg, at least about 550 mg at least about 560 mg, at least about 570 mg, at least about 580 mg, at least about 590 mg, or at least about 600 mg. In certain embodiments, the anti-PD-1 antibody is administered at a flat dose of about 240 mg, about 360 mg, about 480 mg, or about 560 mg. In one particular embodiment, the anti-PD-1 antibody (e.g., nivolumab) is administered at a flat dose of about 360 mg. In another embodiment, the anti-PD-1 antibody (e.g., nivolumab) is administered at a flat dose of about 240 mg.

In some embodiments, the anti-PD-1 antibody is administered at a weight-based dose. For administration of an anti-PD-1 antibody, the dosage can range from at least about 0.01 mg/kg to at least about 20 mg/kg, from at least about 0.1 mg/kg to at least about 10 mg/kg, from about 0.01 mg/kg to about 5 mg/kg, from about 1 mg/kg to about 5 mg/kg, from about 2 mg/kg to about 5 mg/kg, from about 1 mg/kg to about 3 mg/kg, from about 7.5 mg/kg to about 12.5 mg/kg, or from about 0.1 mg/kg to about 30 mg/kg of the subject's body weight. For example, dosages can be at least about 0.1 mg/kg, at least about 0.3 mg/kg, at least about 1 mg/kg, at least about 2 mg/kg, at least about 3 mg/kg, at least about 5 mg/kg, or at least about 10 mg/kg body weight. In certain embodiments, the dosage of the anti-PD-1 antibody is 3 mg/kg body weight.

In one embodiment, a dosage regimen for an anti-PD-1 antibody comprises about 0.3-1 mg/kg body weight, about 5 mg/kg body weight, about 1-5 mg/kg body weight, or about 1-3 mg/kg body weight via intravenous administration, with the antibody being given every about 14-21 days in up to about 6-week or about 12-week cycles until complete response or confirmed progressive disease. In some embodiments, the antibody treatment, or any combination treatment disclosed herein, is continued for at least about 1 month, at least about 3 months, at least about 6 months, at least about 9 months, at least about 1 year, at least about 18 months, at least about 24 months, at least about 3 years, at least about 5 years, or at least about 10 years.

The dosing schedule is typically designed to achieve exposures that result in sustained receptor occupancy (RO) based on typical pharmacokinetic properties of an antibody. An exemplary treatment regime entails administration once per week, once every 2 weeks, once every 3 weeks, once every 4 weeks, once a month, once every 3-6 months or longer. In certain embodiments, an anti-PD-1 antibody such as nivolumab is administered to the subject once every 2 weeks. In other embodiments, the antibody is administered once every 3 weeks. The dosage and scheduling can change during a course of treatment. The anti-PD-1 antibody can be administered in at least two doses, each of the doses is at an amount of about 0.01 mg/kg to about 5 mg/kg, e.g., about 3 mg/kg, at a dosing interval of every two weeks between the two doses. In some embodiments, the anti-PD-1 antibody is administered in at least three, four, five, six, or seven doses (i.e., multiple doses), each of the doses is at an amount of about 0.01 mg/kg to about 5 mg/kg, e.g., about 3 mg/kg, at a dosing interval of every two weeks between two adjacently given doses. The dosage and scheduling may change during a course of treatment. For example, a dosing schedule for anti-PD-1 monotherapy can comprise administering the antibody: (i) every 2 weeks in 6-week cycles; (ii) every 4 weeks for six dosages, then every three months; (iii) every 3 weeks; or (iv) 3-10 mg/kg once followed by 1 mg/kg every 2-3 weeks. Considering that an IgG4 antibody typically has a half-life of 2-3 weeks, a dosage regimen for an anti-PD-1 antibody of the disclosure comprises 0.3-10 mg/kg body weight, e.g., 1-5 mg/kg body weight, e.g., 1-3 mg/kg body weight via intravenous administration, with the antibody being given every 14-21 days in up to 6-week or 12-week cycles until complete response or confirmed progressive disease.

In particular embodiments, the anti-PD-1 antibody is administered at a dose ranging from at least about 0.1 mg/kg to at least about 10.0 mg/kg body weight once about every 1, 2, or 3 weeks. In further embodiments, the anti-PD-1 antibody (e.g., nivolumab) is administered at a dose of at least about 3 mg/kg body weight once about every 2 weeks. In other embodiments, the anti-PD-1 antibody (e.g., pembrolizumab) is administered at a dose of at least about 200 mg every 3 weeks or 2 mg/kg (up to 200 mg) every three weeks. In some embodiments, the anti-PD-1 antibody (e.g., avelumab) is administered at a dose of 10 mg/kg every two weeks.

In some embodiments, the anti-PD-1 antibody is administered in a fixed dose with the CD-122-biased agonist.

In some embodiments, the anti-PD-1 antibody is administered once about every week, once about every 2 weeks, once about every 3 weeks, once about every 4 weeks, once about every 5 weeks, once about every 6 weeks, or once about every 8 weeks. In some embodiments, the anti-PD-1 antibody is administered once about every 2 weeks. In some embodiments, the anti-PD-1 antibody is administered once about every 3 weeks.

In certain embodiments, the anti-PD-1 antibody is administered at a flat dose of about 240 mg, about 360 mg, about 480 mg, or about 560 mg about once every 2 weeks or every 3 weeks. In particular embodiments, the anti-PD-1 antibody is administered at a flat dose of about 360 mg about once every 3 weeks. In other embodiments, the anti-PD-1 antibody is administered at a flat dose of about 240 mg about once every 2 weeks.

In some embodiments, the anti-PD-1 antibody is administered for as long as clinical benefit is observed or until unmanageable toxicity or disease progression occurs. In some embodiments, the anti-PD-1 antibody is administered for at least about 1 cycle, at least about 2 cycles, at least about 3 cycles, at least about 4 cycles, at least about 5 cycles, at least about 7 cycles, at least about 10 cycles, at least about 15 cycles, at least about 20 cycles, or at least about 25 cycles.

In some embodiments, the CD-122-biased agonist (e.g., Formula (I)) is administered as a weight-based dose. In some embodiments, the CD-122-biased agonist (e.g., Formula (I)) is administered at a dose ranging from at least about 0.0001 mg/kg to at least about 0.1 mg/kg body weight. In some embodiments, the CD-122-biased agonist (e.g., Formula (I)) is administered at a dose ranging from at least about 0.001 mg/kg to at least about 0.01 mg/kg body weight. In some embodiments, the CD-122-biased agonist (e.g., Formula (I)) is administered at a dose ranging from at least about 0.003 mg/kg to at least about 0.009 mg/kg body weight. In some embodiments, the CD-122-biased agonist (e.g., Formula (I)) is administered at a dose of about 0.003 mg/kg, about 0.004 mg/kg, about 0.005 mg/kg, about 0.006 mg/kg, about 0.007 mg/kg, about 0.008 mg/kg, about 0.009 mg/kg, or about 0.01 mg/kg body weight protein equivalents. In certain embodiments, the CD-122-biased agonist (e.g., Formula (I)) is administered at a dose of about 0.003 mg/kg body weight. In other embodiments, the CD-122-biased agonist (e.g., Formula (I)) is administered at a dose of about 0.006 mg/kg body weight. In other embodiments, the CD-122-biased agonist (e.g., Formula (I)) is administered at a dose of about 0.009 mg/kg body weight.

In some embodiments, the CD-122-biased agonist (e.g., Formula (I)) is administered once about every week, once about every 2 weeks, once about every 3 weeks, once about every 4 weeks, once about every 5 weeks, once about every 6 weeks, or once about every 8 weeks. In some embodiments, the CD-122-biased agonist (e.g., Formula (I)) is administered once about every 2 weeks. In some embodiments, the CD-122-biased agonist (e.g., Formula (I)) is administered once about every 3 weeks.

In some embodiments, the CD-122-biased agonist (e.g., Formula (I)) is administered at a dose of about 0.003 mg/kg body weight about every 2 weeks. In some embodiments, the CD-122-biased agonist (e.g., Formula (I)) is administered at a dose of about 0.006 mg/kg body weight about every 2 weeks. In some embodiments, the CD-122-biased agonist (e.g., Formula (I)) is administered at a dose of about 0.006 mg/kg body weight about every 3 weeks. In some embodiments, the CD-122-biased agonist (e.g., Formula (I)) is administered at a dose of about 0.009 mg/kg body weight about every 3 weeks.

In certain embodiments, the anti-PD-1 antibody (e.g., nivolumab) is administered at a dose of about 360 mg every 3 weeks and the CD-122-biased agonist (e.g., Formula (I)) is administered at a dose of about 0.006 mg/kg body weight about every 3 weeks. In some embodiments, the anti-PD-1 antibody (e.g., nivolumab) is administered at a dose of about 240 mg every 2 weeks and the CD-122-biased agonist (e.g., Formula (I)) is administered at a dose of about 0.006 mg/kg body weight about every 3 weeks. In some embodiments, the anti-PD-1 antibody (e.g., nivolumab) is administered at a dose of about 240 mg every 2 weeks and the CD-122-biased agonist (e.g., Formula (I)) is administered at a dose of about 0.003 mg/kg body weight about every 3 weeks. In some embodiments, the anti-PD-1 antibody (e.g., nivolumab) is administered at a dose of about 240 mg every 2 weeks and the CD-122-biased agonist (e.g., Formula (I)) is administered at a dose of about 0.006 mg/kg body weight about every 2 weeks. In some embodiments, the anti-PD-1 antibody (e.g., nivolumab) is administered at a dose of about 360 mg every 3 weeks and the CD-122-biased agonist (e.g., Formula (I)) is administered at a dose of about 0.009 mg/kg body weight about every 3 weeks.

In some embodiments, a subtherapeutic dose of the CD-122-biased agonist (e.g., Formula (I)) is used in the methods herein. In some embodiments, a subtherapeutic dose of the anti-PD-1 antibody (e.g., nivolumab) is used in the methods herein. In certain embodiments, the anti-PD-1 antibody (e.g., nivolumab) and the CD-122-biased agonist (e.g., Formula (I)) are each administered at a subtherapeutic dose.

In some embodiments, the anti-PD-1 antibody and the CD-122-biased agonist are formulated for intravenous administration. In certain embodiments, the anti-PD-1 antibody and the CD-122-biased agonist are administered sequentially. In certain embodiments, the anti-PD-1 antibody and the CD-122-biased agonist are administered within 30 minutes of each other. In one embodiment, the anti-PD-1 antibody or is administered before the CD-122-biased agonist. In another embodiment, the CD-122-biased agonist is administered before the anti-PD-1 antibody. In another embodiment, the anti-PD-1 antibody and CD-122-biased agonist are administered concurrently in separate compositions. In a further embodiment, the anti-PD-1 antibody and CD-122-biased agonist are admixed as a single composition for concurrent administration.

Melanoma

Certain aspects of the present disclosure are directed to methods of treating a subject afflicted with a tumor derived from a melanoma comprising administering to the subject: (a) an antibody that binds specifically to a Programmed Death-1 (PD-1) receptor and inhibits PD-1 activity (“anti-PD-1 antibody”); and (b) a CD-122-biased agonist. Melanoma (MEL) is a malignant tumor of melanocytes, the melanin-producing cells found predominantly in skin. Though less common than other skin cancers, it is the most dangerous of skin cancers if not diagnosed early and causes the majority (75%) of skin cancer deaths. The incidence of MEL is increasing worldwide in Caucasian populations, especially where peoples with low amounts of skin pigmentation receive excessive ultraviolet light exposure from the sun. In Europe, the incidence rate is <10-20 per 100,000 population; in the USA 20-30 per 100,000; and in Australia, where the highest incidence is observed, 50-60 per 100,000 (Garbe et al., Eur. J. Cancer. 48(15):2375-90 (2012)). MEL accounts for about 5% of all new cases of cancer in the United States (U.S.), and the incidence continues to rise by almost 3% per year. This translates to an estimated 76,690 new cases in the U.S. in 2013 with 9,480 associated deaths (Siegel et al., CA Cancer J. Clin. 63(1):11-30 (2013)).

For in situ (stage 0) or early-stage MEL (Stages I-II), surgical excision is the primary treatment. In general, the prognosis is excellent for patients with localized disease and tumors 1.0 mm or less in thickness, with 5-year survival rates of more than 90% (NCCN GUIDELINES®, 2013—Melanoma). Where surgical excision is not feasible for in situ melanoma due to comorbidity or cosmetically sensitive tumor location, topical imiquimod (INN) and radiotherapy are emerging as treatments, especially for lentigo maligna. Chemotherapeutic agents for treating MEL include dacarbazine, temozolomide and imatinib for melanoma with a c-KIT mutation, high-dose interleukin-2, and paclitaxel with or without carboplatin. However, these treatments have modest success, with response rates below 20% in first-line (1L) and second-line (2L) settings.

For patients with localized melanomas more than 1.0 mm in thickness, survival rates range from 50-90%. The likelihood of regional nodal involvement increases with increasing tumor thickness. With Stage III MEL (clinically positive nodes and/or in-transit disease), 5-year survival rates range from 20-70%. By far the most lethal is Stage IV MEL where long-term survival in patients with distant metastatic melanoma is less than 10% (NCCN GUIDELINES®, 2013—melanoma).

The types of melanoma that can be treated with the present methods include, but are not limited to, lentigo maligna, lentigo maligna melanoma, superficial spreading melanoma, acral lentiginous melanoma, nucosal melanoma, nodular melanoma, polypoid melanoma, desmoplastic melanoma, amelanotic melanoma, soft-tissue melanoma, melanoma with small nevus-like cells, melanoma with features of a Spitz nevus, or uveal melanoma. The stages of melanoma that can be treated with the present methods include, but are not limited to, (i) Stage I/II (invasive melanoma): T1a characterized by less than 1.0 mm primary tumor thickness, without ulceration, and mitosis <1/mm²; T1b characterized by less than 1.0 mm primary tumor thickness, with ulceration or mitoses ≥1/mm²; T2a characterized by 1.01-2.0 mm primary tumor thickness, without ulceration; (ii) Stage II (high risk melanoma): T2b characterized by 1.01-2.0 mm primary tumor thickness, with ulceration; T3a characterized by 2.01-4.0 mm primary tumor thickness, without ulceration; T3b characterized by 2.01-4.0 mm primary tumor thickness, with ulceration; T4a characterized by greater than 4.0 mm primary tumor thickness, without ulceration; or T4b characterized by greater than 4.0 mm primary tumor thickness, with ulceration; (iii) Stage III (regional metastasis): N1 characterized by single positive lymph node; N2 characterized by two to three positive lymph nodes or regional skin/in-transit metastasis; or N3 characterized by four positive lymph nodes or one lymph node and regional skin/in-transit metastases; and (iv) Stage IV (distant metastasis): M1a characterized by distant skin metastasis, normal LDH; M1b characterized by Lung metastasis, normal LDH; or M1c characterized by other distant metastasis or any distant metastasis with elevated LDH.

Renal Cell Carcinoma (RCC)

Certain aspects of the present disclosure are directed to methods of treating a subject afflicted with a tumor derived from a renal cell carcinoma (RCC) comprising administering to the subject: (a) an antibody that binds specifically to a Programmed Death-1 (PD-1) receptor and inhibits PD-1 activity (“anti-PD-1 antibody”); and (b) a CD-122-biased agonist. RCCs are among the most common tumors to show spontaneous regression (Elhilali et al. (2000) BJU Int 86:613-8; Inman et al. (2013) Eur Urol 63:881-9) while the traditional chemotherapy and radiotherapy had proven disappointing. Stage I RCC is characterized by a tumor that is 7 centimeters or smaller and is found only in the kidney. In stage II, however, the tumor can be larger than 7 centimeters and is found only in the kidney. In stage III, the tumor can be any size and cancer is found only in the kidney and in one or more nearby lymph nodes; or cancer is found in the main blood vessels of the kidney or in the layer of fatty tissue around the kidney. Cancer may also be found in one or more nearby lymph nodes. In stage IV, cancer has been spread beyond the layer of fatty tissue around the kidney and may be found in the adrenal gland above the kidney with cancer, or in nearby lymph nodes; or to other organs, such as the lungs, liver, bones, or brain, and may have spread to lymph nodes. In other embodiments, RCC that is treatable by the present methods is a recurrent RCC.

Non-Small Cell Lung Cancer (NSCLC)

Certain aspects of the present disclosure are directed to methods of treating a subject afflicted with a tumor derived from non-small cell lung cancer (NSCLC) comprising administering to the subject: (a) an antibody that binds specifically to a Programmed Death-1 (PD-1) receptor and inhibits PD-1 activity (“anti-PD-1 antibody”); and (b) a CD-122-biased agonist. NSCLC is the leading cause of cancer death in the U.S. and worldwide, exceeding breast, colon and prostate cancer combined. In the U.S., an estimated 228,190 new cases of lung and bronchial will be diagnosed in the U.S., and some 159,480 deaths will occur because of the disease (Siegel et al. (2014) CA Cancer J Clin 64(1):9-29). The majority of patients (approximately 78%) are diagnosed with advanced/recurrent or metastatic disease. Metastases to the adrenal gland from lung cancer are a common occurrence, with about 33% of patients having such metastases. NSCLC therapies have incrementally improved OS, but benefit has reached a plateau (median OS for late stage patients is just 1 year). Progression after 1L therapy occurred in nearly all of these subjects and the 5-year survival rate is only 3.6% in the refractory setting. From 2005 to 2009, the overall 5-year relative survival rate for lung cancer in the U.S. was 15.9% (NCCN GUIDELINES®, Version 3.2014—Non-Small Cell Lung Cancer, available at: www.nccn.org/professionals/physician_gls/pdf/nscl.pdf, last accessed May 14, 2014).

The present methods can treat a tumor at any stage. In certain embodiments, the tumor is derived from an NSCLC of any stage. There are at least seven stages used for NSCLC: occult (hidden) stage, Stage 0 (carcinoma in situ), Stage I, Stage II, Stage IIIA, Stage IIIB, and Stage IV. In the occult stage, the cancer cannot be seen by imaging or bronchoscopy. In Stage 0, cancer cells are found in the lining of the airways.

In one embodiment, the present methods treat a Stage I non-squamous NSCLC. Stage I NSCLC is divided in Stage IA and IB. In Stage IA, the tumor is in the lung only and is 3 centimeters or smaller. In Stage IB, the cancer has not spread to the lymph nodes and one or more of the following is true: 1) the tumor is larger than 3 centimeters but not larger than 5 centimeters; 2) the cancer has spread to the main bronchus and is at least 2 centimeters below where the trachea joins the bronchus; 3) cancer has spread to the innermost layer of the membrane that covers the lung; or 4) part of the lung has collapsed or developed pneumonitis (inflammation of the lung) in the area where the trachea joins the bronchus.

In another embodiment, the methods of the present disclosure treat a Stage II non-squamous NSCLC. Stage II NSCLC is divided into Stage IIA and IIB. In Stage IIA, the cancer has either spread to the lymph nodes or not. If the cancer has spread to the lymph nodes, then the cancer can only have spread to the lymph nodes on the same side of the chest as the tumor, the lymph nodes with cancer or within the lung or near the bronchus. and one or more of the following is true: 1) the tumor is not larger than 5 centimeters; 2) the cancer has spread to the main bronchus and is at least 2 centimeters below where the trachea joins the bronchus; 3) cancer has spread to the innermost layer of the membrane that covers the lung; or 4) part of the lung has collapsed or developed pneumonitis (inflammation of the lung) in the area where the trachea joins the bronchus. The tumor is also considered Stage IIA if the cancer has not spread to the lymph nodes and one or more of the following is true: 1) the tumor is larger than 5 centimeters but not larger than 7 centimeters; 2) the cancer has spread to the main bronchus and is at least 2 centimeters below where the trachea joins the bronchus; 3) cancer has spread to the innermost layer of the membrane that covers the lung; or 4) part of the lung has collapsed or developed pneumonitis (inflammation of the lung) in the area where the trachea joins the bronchus. In stage IIB, the cancer has either spread to the lymph nodes or not. If the cancer has spread to the lymph nodes, then the cancer can only have spread to the lymph nodes on the same side of the chest as the tumor, the lymph nodes with cancer are within the lung or near the bronchus and one or more of the following is true: 1) the tumor is larger than 5 centimeters but not larger than 7 centimeters; 2) the cancer has spread to the main bronchus and is at least 2 centimeters below where the trachea joins the bronchus; 3) cancer has spread to the innermost layer of the membrane that covers the lung; or 4) part of the lung has collapsed or developed pneumonitis (inflammation of the lung) in the area where the trachea joins the bronchus. The tumor is also considered Stage IIB if the cancer has not spread to the lymph nodes and one or more of the following is true: 1) the tumor is larger than 7 centimeters; 2) the cancer has spread to the main bronchus (and is at least 2 centimeters below where the trachea joins the bronchus), the chest wall, the diaphragm, or the nerve that controls the diaphragm; 3) cancer has spread to the membrane around the heart or lining the chest wall; 4) the whole lung has collapsed or developed pneumonitis (inflammation of the lung); or 5) there are one or more separate tumors in the same lobe of the lung.

In other embodiments, any methods of the present disclosure treats Stage III non-squamous NSCLC. Stage IIIA is divided into 3 sections. These 3 sections are based on 1) the size of the tumor; 2) where the tumor is found and 3) which (if any) lymph nodes have cancer. In the first type of Stage IIIA NSCLC, the cancer has spread to the lymph nodes on the same side of the chest as the tumor, and the lymph nodes with the cancer are near the sternum or where the bronchus enters the lung. Additionally: 1) the tumor can be any size; 2) part of the lung (where the trachea joins the bronchus) or the whole lung can have collapsed or developed pneumonitis (inflammation of the lung); 3) there can be one or more separate tumors in the same lobe of the lung; and 4) cancer can have spread to any of the following: a) main bronchus, but not the area where the trachea joins the bronchus, b) chest well, c) diaphragm and the nerve that controls it, d) membrane around the lung or lining the chest wall, e) membrane around the heart. In the second type of Stage IIIA NSCLC, the cancer has spread to the lymph nodes on the same side of the chest as the tumor, and the lymph nodes with the cancer are within the lung or near the bronchus. Additionally: 1) the tumor can be any size; 2) the whole lung can have collapsed or developed pneumonitis (inflammation of the lung); 3) there can be one or more separate tumors in the any of the lobes of the lung with cancer; and 4) cancer can have spread to any of the following: a) main bronchus, but not the area where the trachea joins the bronchus, b) chest well, c) diaphragm and the nerve that controls it, d) membrane around the lung or lining the chest wall, e) heart or the membrane around it, f) major blood vessels that lead to or from the heart, g) trachea, h) esophagus, i) nerve that controls the larynx (voice box), j) sternum (chest bone) or backbone, or k) carina (where the trachea joins the bronchi). In the third type of Stage IIIA NSCLC, the cancer has not spread to the lymph nodes, the tumor can be any size, and cancer has spread to any one of the following: a) heart, b) major blood vessels that lead to or from the heart, c) trachea, d) esophagus, e) nerve that controls the larynx (voice box), f) sternum (chest bone) or backbone, or g) carina (where the trachea joins the bronchi). Stage IIIB is divided into 2 sections depending on 1) the size of the tumor, 2) where the tumor is found, and 3) which lymph nodes have cancer. In the first type of Stage IIIB NSCLC, the cancer has spread to the lymph nodes on the opposite side of the chest as the tumor. Additionally, 1) the tumor can be any size; 2) part of the lung (where the trachea joins the bronchus) or the whole lung can have collapsed or developed pneumonitis (inflammation of the lung); 3) there can be one or more separate tumors in any of the lobs of the lung with cancer; and 4) cancer can have spread to any of the following: a) main bronchus, b) chest well, c) diaphragm and the nerve that controls it, d) membrane around the lung or lining the chest wall, e) heart or the membrane around it, f) major blood vessels that lead to or from the heart, g) trachea, h) esophagus, i) nerve that controls the larynx (voice box), j) sternum (chest bone) or backbone, or k) carina (where the trachea joins the bronchi). In the second type of Stage IIIB NSCLC, the cancer has spread to lymph nodes on the same side of the chest as the tumor. The lymph nodes with cancer are near the sternum (chest bone) or where the bronchus enters the lung. Additionally, 1) the tumor can be any size; 2) there can be separate tumors in different lobes of the same lung; and 3) cancer has spread to any of the following: a) heart, b) major blood vessels that lead to or from the heart, c) trachea, d) esophagus, e) nerve that controls the larynx (voice box), f) sternum (chest bone) or backbone, or g) carina (where the trachea joins the bronchi).

In some embodiments, the methods of the disclosure treat a Stage IV non-squamous NSCLC. In Stage IV NSCLC, the tumor can be any size and the cancer can have spread to the lymph nodes. One or more of the following is true in Stage IV NSCLC: 1) there are one or more tumors in both lungs; 2) cancer is found in the fluid around the lungs or heart; and 3) cancer has spread to other parts of the body, such as the brain, liver, adrenal glands, kidneys or bone.

In some embodiments, the subject has never smoked. In certain embodiments, the subject formerly smoked. In one embodiments, the subject currently smokes. In certain embodiments, the subject has cancer cells that are squamous. In certain embodiments, the subject has cancer cells that are non-squamous.

Urothelial Carcinoma (UC)

Certain aspects of the present disclosure are directed to methods of treating a subject afflicted with a tumor derived from an urothelial carcinoma (UC) comprising administering to the subject: (a) an antibody that binds specifically to a Programmed Death-1 (PD-1) receptor and inhibits PD-1 activity (“anti-PD-1 antibody”); and (b) a CD-122-biased agonist. In certain embodiments, the UC comprises a bladder carcinoma. In other embodiments, the UC comprises a carcinoma of the ureters. In still other embodiments, the UC comprises a carcinoma of the renal pelvis. In certain embodiments, the UC comprises a carcinoma of any one or more of the bladder, the ureters, and the renal pelvis.

In some embodiments, the UC comprises a transitional cell carcinoma.

Transitional cell carcinomas arise from the urothelial cells lining the inside of the bladder, the ureters, and the renal pelvis.

In some embodiments, the UC comprises a squamous cell carcinoma. A squamous cell carcinoma, e.g., of the bladder, arises from the bladder uroepithelium with pure squamous phenotype.

In some embodiments, the UC comprises an adenocarcinoma. An adenocarcinoma, e.g., of the bladder, is defined as a tumor composed entirely of malignant glandular epithelium.

In certain embodiments, the UC or cancer derived therefrom comprises a bladder carcinoma, a carcinoma of the ureters, a carcinoma of the renal pelvis, a transitional cell carcinoma, a squamous cell cancer, an adenocarcinoma, or any combination thereof.

PD-L1 Expression Status

The PD-L1 status of a tumor in a subject can be measured prior to administering any composition or utilizing any method disclosed herein. PD-L1 expression can be determined by any methods known in the art.

In order to assess the PD-L1 expression, in one embodiment, a test tissue sample can be obtained from the patient who is in need of the therapy. In another embodiment, the assessment of PD-L1 expression can be achieved without obtaining a test tissue sample. In some embodiments, selecting a suitable patient includes (i) optionally providing a test tissue sample obtained from a patient with cancer of the tissue, the test tissue sample comprising tumor cells and/or tumor-infiltrating inflammatory cells; and (ii) assessing the proportion of cells in the test tissue sample that express PD-L1 on the surface of the cells based on an assessment that the proportion of cells in the test tissue sample that express PD-L1 on the cell surface is higher than a predetermined threshold level.

In any of the methods comprising the measurement of PD-L1 expression in a test tissue sample, however, it should be understood that the step comprising the provision of a test tissue sample obtained from a patient is an optional step. It should also be understood that in certain embodiments the “measuring” or “assessing” step to identify, or determine the number or proportion of, cells in the test tissue sample that express PD-L1 on the cell surface is performed by a transformative method of assaying for PD-L1 expression, for example by performing a reverse transcriptase-polymerase chain reaction (RT-PCR) assay or an IHC assay. In certain other embodiments, no transformative step is involved and PD-L1 expression is assessed by, for example, reviewing a report of test results from a laboratory. In certain embodiments, the steps of the methods up to, and including, assessing PD-L1 expression provides an intermediate result that may be provided to a physician or other healthcare provider for use in selecting a suitable candidate for the anti-PD-1 antibody or anti-PD-L1 antibody therapy. In certain embodiments, the steps that provide the intermediate result is performed by a medical practitioner or someone acting under the direction of a medical practitioner. In other embodiments, these steps are performed by an independent laboratory or by an independent person such as a laboratory technician.

In certain embodiments of any of the present methods, the proportion of cells that express PD-L1 is assessed by performing an assay to determine the presence of PD-L1 RNA. In further embodiments, the presence of PD-L1 RNA is determined by RT-PCR, in situ hybridization or RNase protection. In other embodiments, the proportion of cells that express PD-L1 is assessed by performing an assay to determine the presence of PD-L1 polypeptide. In further embodiments, the presence of PD-L1 polypeptide is determined by immunohistochemistry (IHC), enzyme-linked immunosorbent assay (ELISA), in vivo imaging, or flow cytometry. In some embodiments, PD-L1 expression is assayed by IHC. In other embodiments of all of these methods, cell surface expression of PD-L1 is assayed using, e.g., IHC or in vivo imaging. Chen et al., (2013) Clin Cancer Res 19(13): 3462-3473.

Imaging techniques have provided important tools in cancer research and treatment. Recent developments in molecular imaging systems, including positron emission tomography (PET), single-photon emission computed tomography (SPECT), fluorescence reflectance imaging (FM), fluorescence-mediated tomography (FMT), bioluminescence imaging (BLI), laser-scanning confocal microscopy (LSCM) and multiphoton microscopy (MPM), will likely herald even greater use of these techniques in cancer research. Some of these molecular imaging systems allow clinicians to not only see where a tumor is located in the body, but also to visualize the expression and activity of specific molecules, cells, and biological processes that influence tumor behavior and/or responsiveness to therapeutic drugs (Condeelis and Weissleder, “In vivo imaging in cancer,” Cold Spring Harb. Perspect. Biol. 2(12):a003848 (2010)). Antibody specificity, coupled with the sensitivity and resolution of PET, makes immunoPET imaging particularly attractive for monitoring and assaying expression of antigens in tissue samples (McCabe and Wu, “Positive progress in immunoPET—not just a coincidence,” Cancer Biother. Radiopharm. 25(3):253-61 (2010); Olafsen et al., “ImmunoPET imaging of B-cell lymphoma using 124I-anti-CD20 scFv dimers (diabodies),” Protein Eng. Des. Sel. 23(4):243-9 (2010)). In certain embodiments of any of the present methods, PD-L1 expression is assayed by immunoPET imaging. In certain embodiments of any of the present methods, the proportion of cells in a test tissue sample that express PD-L1 is assessed by performing an assay to determine the presence of PD-L1 polypeptide on the surface of cells in the test tissue sample. In certain embodiments, the test tissue sample is a FFPE tissue sample. In other embodiments, the presence of PD-L1 polypeptide is determined by IHC assay. In further embodiments, the IHC assay is performed using an automated process. In some embodiments, the IHC assay is performed using an anti-PD-L1 monoclonal antibody to bind to the PD-L1 polypeptide.

In one embodiment of the present methods, an automated IHC method is used to assay the expression of PD-L1 on the surface of cells in FFPE tissue specimens. This disclosure provides methods for detecting the presence of human PD-L1 antigen in a test tissue sample, or quantifying the level of human PD-L1 antigen or the proportion of cells in the sample that express the antigen, which methods comprise contacting the test sample, and a negative control sample, with a monoclonal antibody that specifically binds to human PD-L1, under conditions that allow for formation of a complex between the antibody or portion thereof and human PD-L1. In certain embodiments, the test and control tissue samples are FFPE samples. The formation of a complex is then detected, wherein a difference in complex formation between the test sample and the negative control sample is indicative of the presence of human PD-L1 antigen in the sample. Various methods are used to quantify PD-L1 expression.

In a particular embodiment, the automated IHC method comprises: (a) deparaffinizing and rehydrating mounted tissue sections in an autostainer; (b) retrieving antigen using a decloaking chamber and pH 6 buffer, heated to 110° C. for 10 min; (c) setting up reagents on an autostainer; and (d) running the autostainer to include steps of neutralizing endogenous peroxidase in the tissue specimen; blocking non-specific protein-binding sites on the slides; incubating the slides with primary antibody; incubating with a post primary blocking agent; incubating with NovoLink Polymer; adding a chromogen substrate and developing; and counterstaining with hematoxylin.

For assessing PD-L1 expression in tumor tissue samples, a pathologist examines the number of membrane PD-L1⁺ tumor cells in each field under a microscope and mentally estimates the percentage of cells that are positive, then averages them to come to the final percentage. The different staining intensities are defined as 0/negative, 1+/weak, 2+/moderate, and 3+/strong. Typically, percentage values are first assigned to the 0 and 3+ buckets, and then the intermediate 1+ and 2+ intensities are considered. For highly heterogeneous tissues, the specimen is divided into zones, and each zone is scored separately and then combined into a single set of percentage values. The percentages of negative and positive cells for the different staining intensities are determined from each area and a median value is given to each zone. A final percentage value is given to the tissue for each staining intensity category: negative, 1+, 2+, and 3+. The sum of all staining intensities needs to be 100%. In one embodiment, the threshold number of cells that needs to be PD-L1 positive is at least about 100, at least about 125, at least about 150, at least about 175, or at least about 200 cells. In certain embodiments, the threshold number or cells that needs to be PD-L1 positive is at least about 100 cells.

Staining is also assessed in tumor-infiltrating inflammatory cells such as macrophages and lymphocytes. In most cases macrophages serve as an internal positive control since staining is observed in a large proportion of macrophages. While not required to stain with 3+ intensity, an absence of staining of macrophages should be taken into account to rule out any technical failure. Macrophages and lymphocytes are assessed for plasma membrane staining and only recorded for all samples as being positive or negative for each cell category. Staining is also characterized according to an outside/inside tumor immune cell designation. “Inside” means the immune cell is within the tumor tissue and/or on the boundaries of the tumor region without being physically intercalated among the tumor cells. “Outside” means that there is no physical association with the tumor, the immune cells being found in the periphery associated with connective or any associated adjacent tissue.

In certain embodiments of these scoring methods, the samples are scored by two pathologists operating independently, and the scores are subsequently consolidated. In certain other embodiments, the identification of positive and negative cells is scored using appropriate software.

A histoscore is used as a more quantitative measure of the IHC data. The histoscore is calculated as follows:

Histoscore=[(% tumor×1 (low intensity))+(% tumor×2 (medium intensity))+(% tumor×3 (high intensity)]

To determine the histoscore, the pathologist estimates the percentage of stained cells in each intensity category within a specimen. Because expression of most biomarkers is heterogeneous the histoscore is a truer representation of the overall expression. The final histoscore range is 0 (no expression) to 300 (maximum expression).

An alternative means of quantifying PD-L1 expression in a test tissue sample IHC is to determine the adjusted inflammation score (AIS) score defined as the density of inflammation multiplied by the percent PD-L1 expression by tumor-infiltrating inflammatory cells (Taube et al., “Colocalization of inflammatory response with B7-hl expression in human melanocytic lesions supports an adaptive resistance mechanism of immune escape,” Sci. Transl. Med. 4(127):127ra37 (2012)).

In one embodiment, the PD-L1 expression level of a tumor (e.g., a tumor derived from a NHL and/or a HL) is at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 40%, at least about 50%, at least about 60%, at least about 70%, at least about 75%, at least about 80%, at least about 85%, at least about 90%, at least about 95%, or about 100%. In another embodiment, the PD-L1 status of a tumor is at least about 1%. In other embodiments, the PD-L1 status of a tumor is at least about 5%. In a certain embodiment, the PD-L1 status of a tumor is at least about 10%. In one embodiment, the PD-L1 status of the tumor is at least about 25%. In a particular embodiment, the PD-L1 status of the tumor is at least about 50%.

In some embodiments, “PD-L1 positive” as used herein refers to PD-L1 expression of at least about 1%. In other embodiments, “PD-L1 positive” as used herein refers to PD-L1 expression of at least about 5%. In one embodiment, the PD-L1 positive tumors can thus have at least about 1%, at least about 2%, at least about 5%, at least about 10%, at least about 20%, at least about 25%, at least about 30%, at least about 40%, at least about 50%, at least about 60%, at least about 70%, at least about 75%, at least about 80%, at least about 85%, at least about 90%, at least about 95%, or about 100% of the tumor cells expressing PD-L1 as measured by an automated IHC. In certain embodiments, “PD-L1 positive” means that there are at least 100 cells that express PD-L1 on the surface of the cells.

Pharmaceutical Compositions

Therapeutic agents of the present disclosure can be constituted in a composition, e.g., a pharmaceutical composition containing an antibody and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. In some embodiments, the carrier for a composition containing an antibody is suitable for intravenous, intramuscular, subcutaneous, parenteral, spinal, or epidermal administration (e.g., by injection or infusion). In some embodiments, the subcutaneous injection is based on Halozyme Therapeutics' ENHANZE® drug-delivery technology (see U.S. Pat. No. 7,767,429, which is incorporated by reference herein in its entirety). ENHANZE® uses a co-formulation of an Ab with recombinant human hyaluronidase enzyme (rHuPH20), which removes traditional limitations on the volume of biologics and drugs that can be delivered subcutaneously due to the extracellular matrix (see U.S. Pat. No. 7,767,429). A pharmaceutical composition of the disclosure can include one or more pharmaceutically acceptable salts, anti-oxidants, aqueous and non-aqueous carriers, and/or adjuvants such as preservatives, wetting agents, emulsifying agents, and dispersing agents.

Kits

Also within the scope of the present disclosure are kits comprising an anti-PD-1 antibody and a CD-122-biased agonist for therapeutic uses. Kits typically include a label indicating the intended use of the contents of the kit and instructions for use. The term label includes any writing, or recorded material supplied on or with the kit, or which otherwise accompanies the kit. Accordingly, this disclosure provides a kit for treating a subject afflicted with a cancer, the kit comprising: (a) a dosage ranging from about 10 mg to about 600 mg of an anti-PD-1 antibody; (b) a dosage ranging from about 0.0001 mg to about 0.1 mg of a CD-122-biased agonist; and (c) instructions for using the anti-PD-1 antibody and the CD-122-biased agonist in any of the combination therapy methods disclosed herein. In certain embodiments, the anti-PD-1 antibody and the CD122 agonist can be co-packaged in unit dosage form. In certain embodiments for treating human patients, the kit comprises an anti-human PD-1 antibody disclosed herein, e.g., nivolumab, pembrolizumab, MEDI0680 (formerly AMP-514), AMP-224, or BGB-A317. In other embodiments, the kit comprises a CD-122-biased agonist disclosed herein.

The present disclosure is further illustrated by the following examples which should not be construed as further limiting. The contents of all references cited throughout this application are expressly incorporated herein by reference.

### EXAMPLES

### Example 1

A phase 1b clinical trial was conducted to evaluate the safety and efficacy of a combination therapy comprising an anti-PD-1 antibody (nivolumab) and a CD-122-biased agonist for the treatment of various types of tumors.

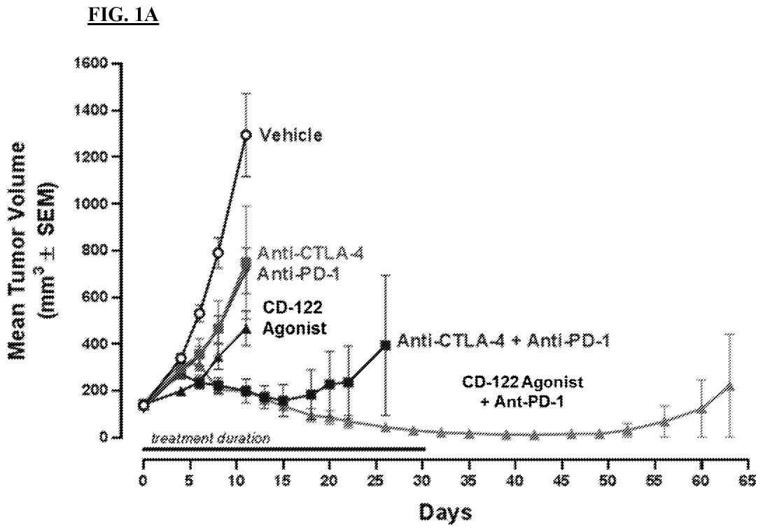

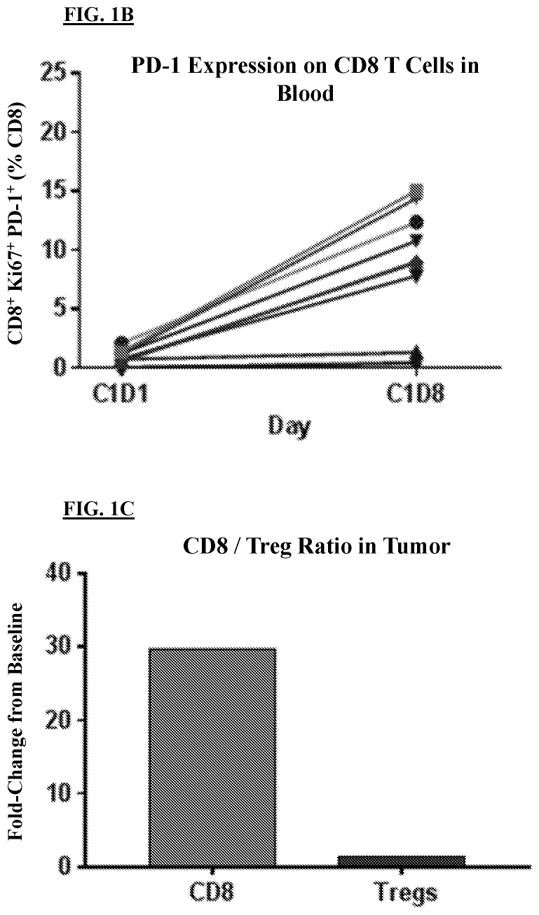

Preclinical data showed that tumor size is reduced following treatment with a combination of a CD-122-biased agonist and an anti-PD-1 antibody (FIG. 1A). This reduction in tumor size is more pronounced and more persistent than the effects on tumor size observed following anti-PD-1 monotherapy, CD-122-biased agonist monotherapy, anti-CTLA-4 monotherapy, and a combination therapy of an anti-PD-1 antibody and an anti-CTLA-4 antibody (FIG. 1A).

A prior clinical trial investigating the effects of CD-122-biased agonist monotherapy revealed that treatment with a CD-122-biased agonist led to increased proliferation of CD8⁺/PD-1⁺ cells in the blood of patients by treatment day 8 (FIG. 1B). CD-122-biased agonist monotherapy was also found to increase the number of CD8⁺ T cells in tumor tissue by nearly 30-fold relative to baseline, without a similar increase in the number of immunosuppressive Treg cells (FIG. 1C).

Study Design

The primary outcome measurements of the trial were to determine the safety and tolerability of the combination therapy of nivolumab and a CD-122-biased agonist; to define the maximum tolerable dose (MTD) and/or recommended phase 2 dose (RP2D); and to assess efficacy by the objective response rate (ORR) at the RP2D. Secondary outcome measures included overall survival (OS) and progression-free survival (PFS). Exploratory objectives included determining the efficacy at the RP2D; assess the immunological effects, and accommodate disease-specific pharmacodynamics markers; measure the pharmacokinetics (PK) of the CD-122-biased agonist, nivolumab, and metabolites; assess development of anti-drug antibodies; and assess the association between efficacy measures and PD-L1 expression in tumors.

NSCLC patients enrolled in the study had histologically confirmed or cytologically confirmed diagnosis of stage IV NSCLC lacking epidermal growth factor receptor (EGFR)-sensitizing mutation and/or anaplastic lymphoma kinase (ALK) translocation. Patients can have experienced disease recurrence or progression during or after a prior platinum-based chemotherapy-containing regimen for advanced or metastatic disease, or patient refuses standard of care. Patients who received platinum-containing adjuvant, neoadjuvant, or definitive chemoradiation therapy given for locally advanced disease and developed recurrent (local or metastatic) disease within six months of completing therapy are eligible.

NSCLC patients were divided into subpopulation A (immuno-oncology (“I-O”) naïve) and subpopulation B (I-O relapsed/refractory). In subpopulation A, first and second line patients must not have received any prior I-O regimens, including but not limited to checkpoint inhibitors such as anti-PD-1, anti-PD-L1, anti-PD-L2, anti-CD137, or anti-CTLA-4 antibody, or any other antibody or drug specifically targeting T cell co-stimulation or checkpoint pathways, indoleamine 2, 3-dioxygenase pathway inhibitors, cancer vaccines, adoptive-cell therapies, or other cytokine therapies. In subpopulation B, second and third line patients must have received only one prior line of therapy with a checkpoint inhibitor (anti-PD-1 or anti-PD-L1) alone or in combination with chemotherapy, which must be their most recent anti-cancer treatment. Patients must have documented disease progression during treatment or within 24 months of completing treatment with a checkpoint inhibitor.

Remaining subjects must have had histologically confirmed diagnosis of a locally advanced or metastatic renal cell carcinoma, melanoma, non-small cell lung cancer (NSCLC), bladder, or triple negative breast cancer (TNBC). Melanoma patients must have a known BRAF status. I-O relapsed/refractory patients must have documented disease progression during or following treatment with 1 prior line of therapy with anti-PD-1/PD-L1. Patients were excluded if they had a prior IL-2 therapy.

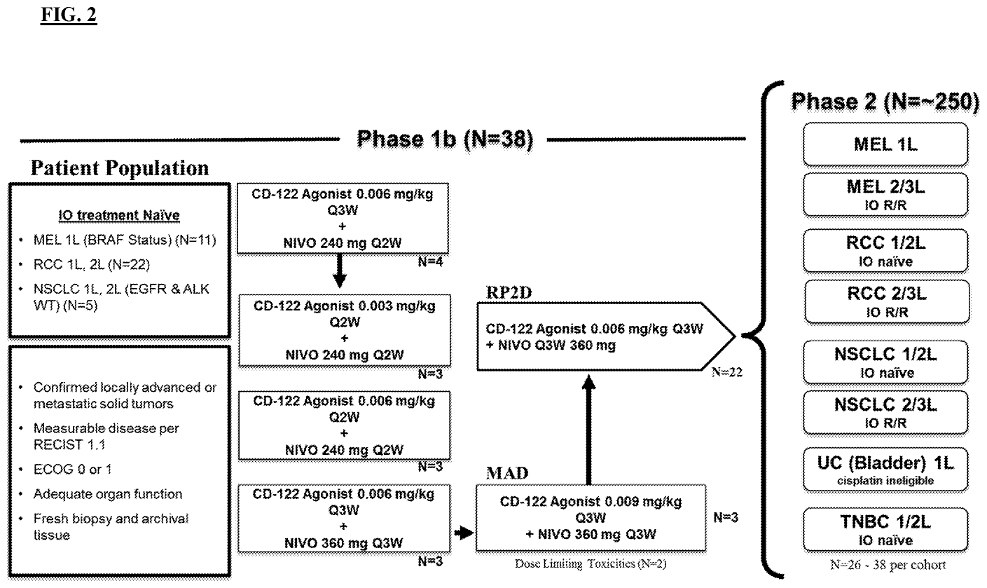

Thirty-eight total patients were enrolled, of which 11 were previously diagnosed with melanoma, 22 were previously diagnosed with RCC, and 5 were previously diagnosed with NSCLC (FIG. 2; Table 1).

TABLE 1
Patient Demographics and Disease Characteristics

| | Total | | Melanoma | | RCC | | NSCLC | |
|---|---|---|---|---|---|---|---|---|
| | (N = 38) | | (N = 11) | | (N = 22) | | (N = 5) | |
| Sex | | | | | | | | |
| Male | 30 | (78.9%) | 7 | (63.6%) | 19 | (86.4%) | 4 | (80.0%) |
| Female | 8 | (21.1%) | 4 | (36.4%) | 3 | (13.6%) | 1 | (20.0%) |
| Age (years) | | | | | | | | |
| Median | 61 | (22-72) | 62 | (22-70) | 61 | (45-72) | 58 | (53-72) |
| (Range) | | | | | | | | |
| ECOG | | | | | | | | |
| Performance | | | | | | | | |
| Status | | | | | | | | |
| 0 | 25 | (65.8%) | 8 | (72.7%) | 15 | (68.2%) | 2 | (40.0%) |
| 1 | 13 | (34.2%) | 3 | (27.3%) | 7 | (31.8%) | 4 | (80.0%) |
| Prior systemic | | | | | | | | |
| therapy for | | | | | | | | |
| metastatic | | | | | | | | |
| disease | | | | | | | | |
| 0 | 26 | (68.4%) | 11 | (100%) | 14 | (63.6%) | 1 | (20.0%) |

| | | | | | | | |
|---|---|---|---|---|---|---|---|
| 1 | 12 | (31.6%) | 0 | 8 | (36.4%) | 4 | (80.0%) |

During the multiple ascending dose (MAD) phase, four patients were administered a combination of the 0.006 mg/kg body weight CD-122-biased agonist (formula I, supra) every 3 weeks and 240 mg of nivolumab every 2 weeks; three patients were administered a combination of the 0.003 mg/kg body weight CD-122-biased agonist every 2 weeks and 240 mg of nivolumab every 2 weeks; three patients were administered a combination of the 0.006 mg/kg body weight CD-122-biased agonist every 2 weeks and 240 mg of nivolumab every 2 weeks; three patients were administered a combination of the 0.006 mg/kg body weight CD-122-biased agonist every 3 weeks and 360 mg of nivolumab every 3 weeks; and three patients were administered a combination of the 0.009 mg/kg body weight CD-122-biased agonist every 3 weeks and 360 mg of nivolumab every 3 weeks. The remaining twenty-two patients received a combination of the 0.006 mg/kg body weight CD-122-biased agonist every 3 weeks and 360 mg of nivolumab every 3 weeks. A planned phase 2 trial will investigate the efficacy of a combination therapy comprising 0.006 mg/kg body weight CD-122-biased agonist every 3 weeks and 360 mg of nivolumab every 3 weeks for the treatment of melanoma, RCC, NSCLC, UC (e.g., bladder cancer), and TNBC.

Efficacy was assessed every eight (±1) weeks per RECIST v1.1 and immune-related RECIST (irRECIST). Per-protocol efficacy-evaluable includes patients with >1 post baseline scan. Adverse events were assessed by Common Terminology Criteria for Adverse Events (CTCAE) v4.03. Safety-evaluable includes >1 dose of study treatment as of data cutoff. Biomarker exploratory analyses were conducted, including identifying baseline PDL1 status by tumor type.

Of the 11 melanoma subjects, 6 (54%) had a mutant V600E BRAF status, and 5 (45.5%) had a positive (>5%) PD-L1 status (Table 2). Of the 22 RCC subjects, 14 were classified as 1L RCC, of which 4 (28.6%) were identified as having a positive (>1%) PD-L1 status (Table 2). Eight of the RCC subjects were classified as 2L RCC, of which 5 (62.5%) were identified as having a positive (>1%) PD-L1 status (Table 2). None of the 5 NSCLC subjects were identified as being PD-L1 positive (>1%) (Table 2).

TABLE 2
Disease Characteristics

| Melanoma | N = 11 | % |
|---|---|---|
| BRAF status | | |
| Mutant V600E | 6 | 54.5 |
| Wild-Type | 5 | 45.5 |
| LDH at baseline* | | |
| High | 4 | 36.4 |
| Normal | 7 | 63.6 |
| PD-L1 status ** | | |
| Positive >5% | 5 | 45.5 |
| Negative | 6 | 54.5 |
| Stage | | |
| M1a | 1 | 9.1 |
| M1b | 2 | 18.2 |
| M1c | 8 | 72.7 |
| Liver metastases at baseline | | |
| Yes | 4 | 36.4 |
| No | 7 | 63.6 |

| RCC | N = 22 | % |
|---|---|---|
| 1L IMDC Score | n = 14 | |
| Favorable | 2 | 14.3 |
| Intermediate | 12 | 85.7 |
| IL PD-L1 status ** | N = 14 | |
| Positive >1% | 4 | 28.6 |
| Negative | 8 | 57.1 |
| No available biopsy | 2 | 14.3 |
| 2L PD-L1 status ** | N = 8 | |
| Positive >1% | 5 | 62.5 |
| Negative | 3 | 37.5 |

| NSCLC | N = 5 | % |
|---|---|---|
| Histologic Subtype | | |
| Adenocarcinoma | 4 | 80.0 |
| Squamous | 1 | 20.0 |
| Smoker | | |
| Yes | 4 | 80.0 |
| Unknown | 1 | 20.0 |
| PD-L1 status ** | | |
| Positive >1% | 0 | 0 |
| Negative | 5 | 100.0 |

*Based on maximum value prior to dosing.
** Measured using either 28-8 or 22C3 assays on fresh or archival tumor with specific cutoffs.

Results

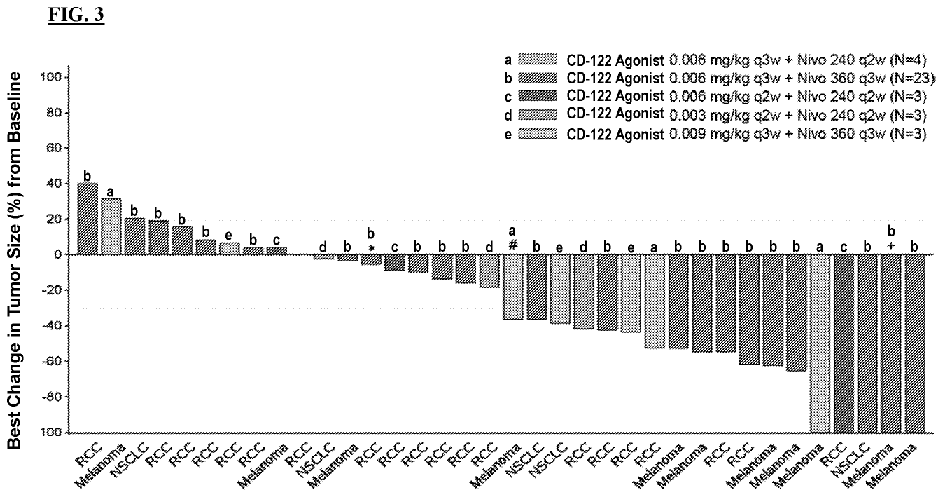

Of all 36 patients treated with a combination of CD-122-biased agonist and Nivolumab, 26 (72%) showed a reduction in the size of the target lesion (FIG. 3). Administration of 0.006 mg/kg CD-122-biased agonist one time every 3 weeks and 360 mg nivolumab administered one time every three weeks was selected as the RP2D (FIGS. 2-3).

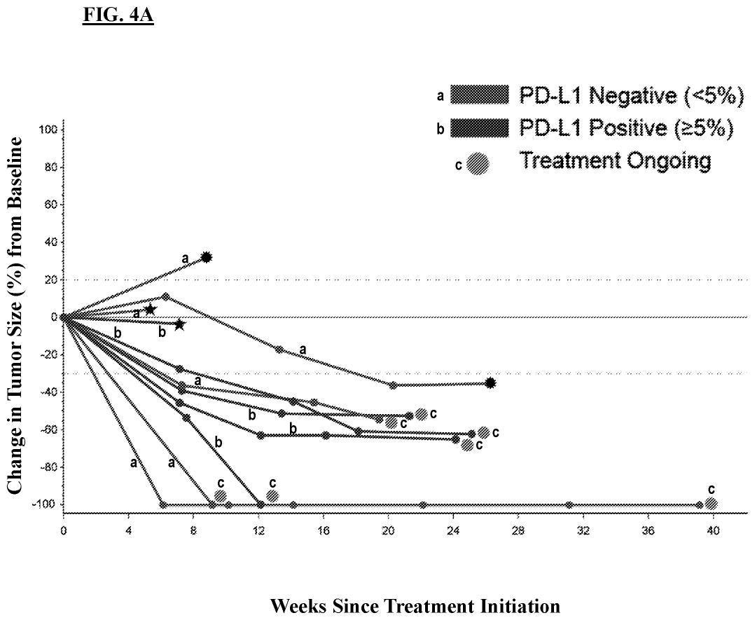

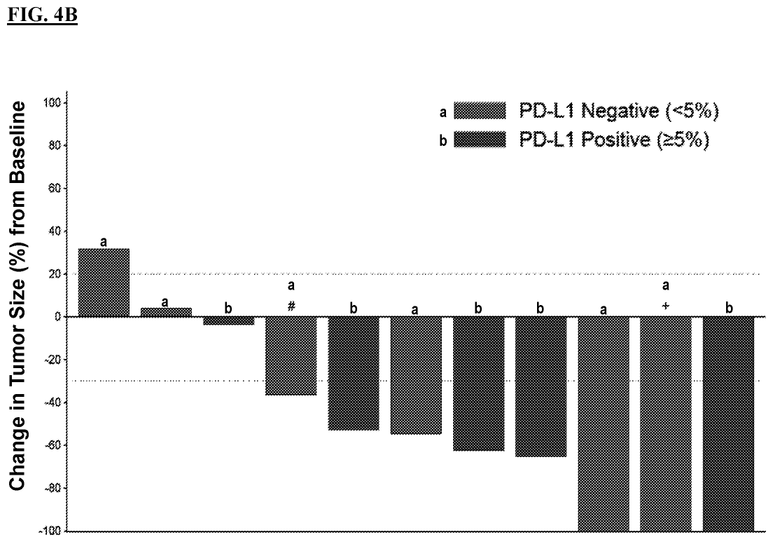

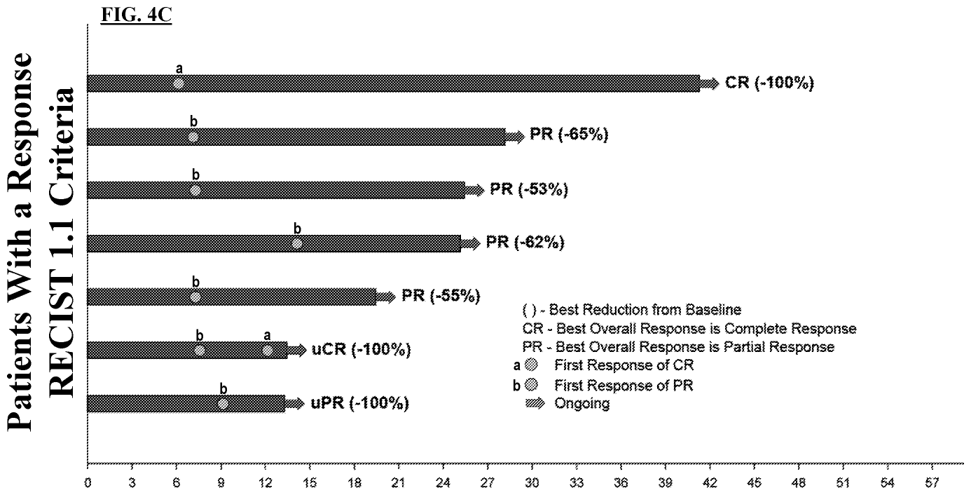

Treatment of melanoma patient with a combination therapy of 0.006 mg/kg CD-122-biased agonist one time every 3 weeks and 360 mg nivolumab administered one time every three weeks yielded a best overall response (ORR) of 7/11 patients (64%), with a disease control rate (DCR; best overall responses of complete response (CR), partial response (PR), or stable disease (SD)) of 10/11 (91%) by RECIST. By iRECIST, the ORR for melanoma patients was 8/11 (73%). The median time to response was 1.7 months. Nine out of 11 melanoma patients experienced a decrease in tumor size, regardless of PD-L1 status (FIGS. 4A-4B). Of the 7 patients having a best overall response, 2 patients exhibited a complete response, and 5 patients exhibited a partial response (FIG. 4C).

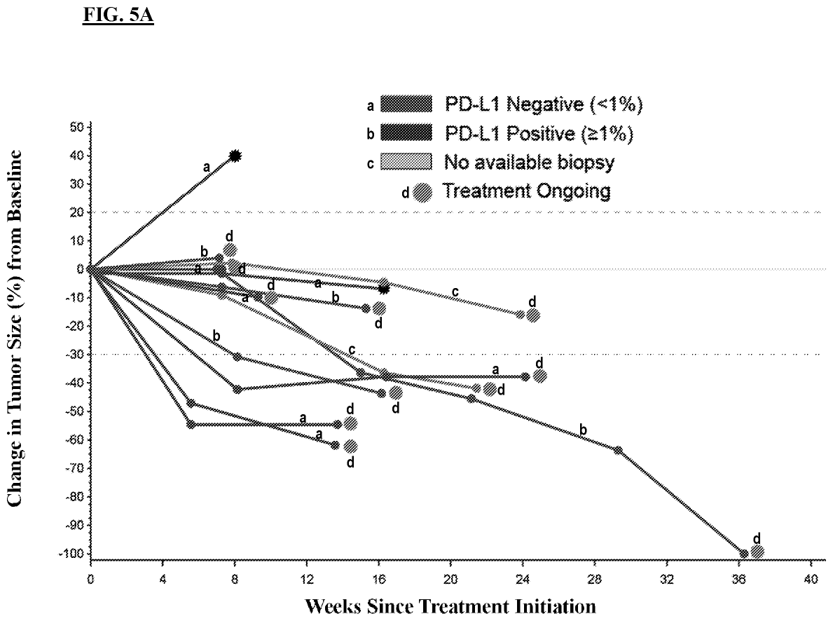

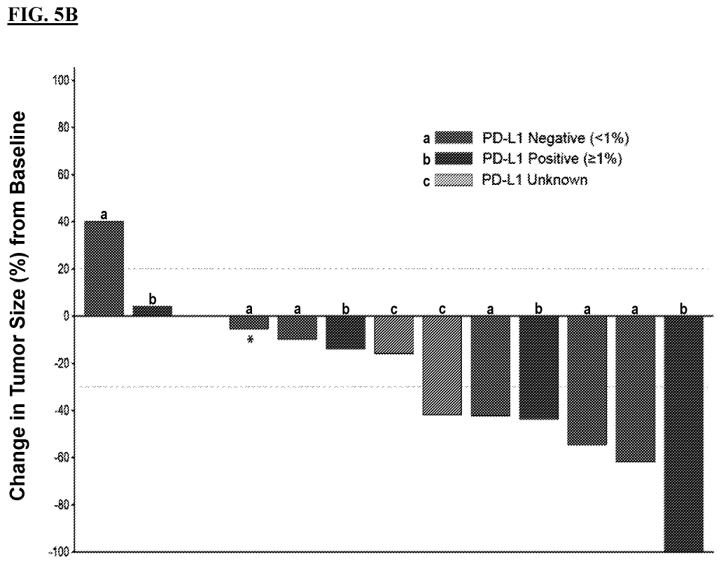

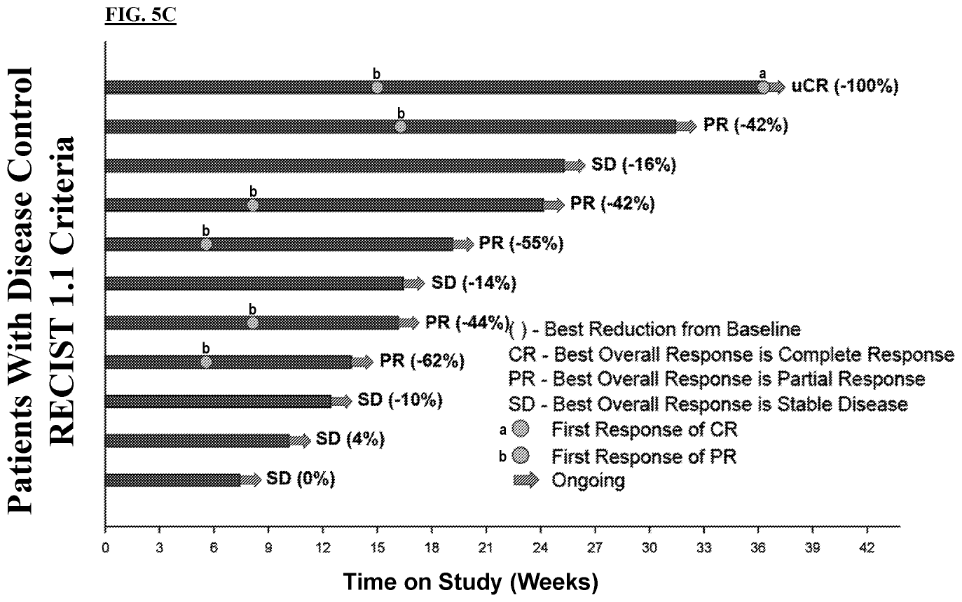

Similar results were observed in first line renal cell carcinoma (RCC 1L) patients, where the best overall response by RECIST was observed in 6/13 (46%) of patients, with 11/13 (85%) experiencing DCR (FIGS. 5A-5B). The median time to response was 1.9 months. Ten of 13 RCC 1L patients experienced a decrease in tumor size, regardless of PD-L1 status (FIGS. 5A-5B). Of the 11 patients exhibiting DCR, 1 patient exhibited a complete response, 5 patients exhibited a partial response, and 5 patients exhibited stable disease (FIG. 5C).

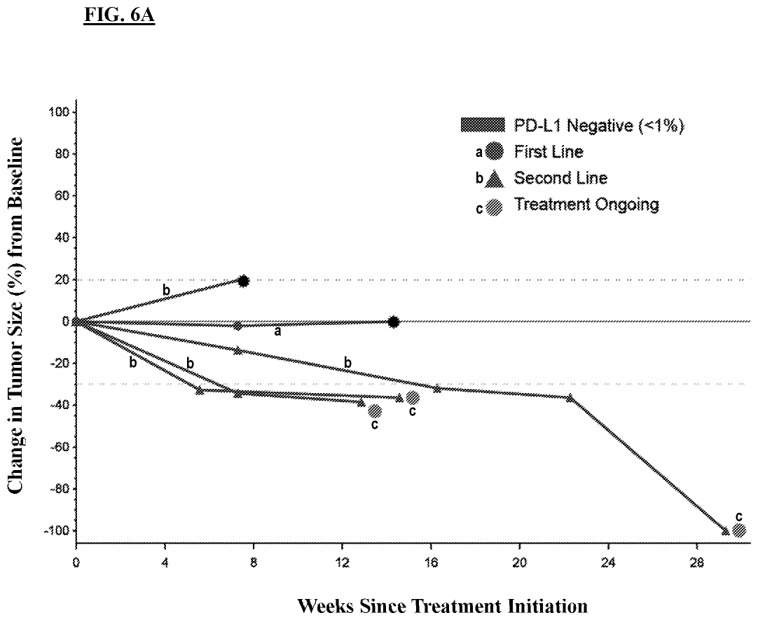

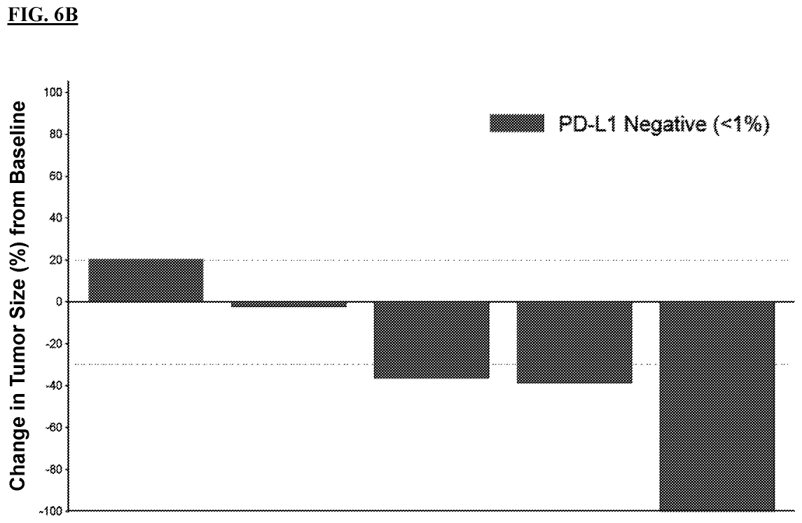

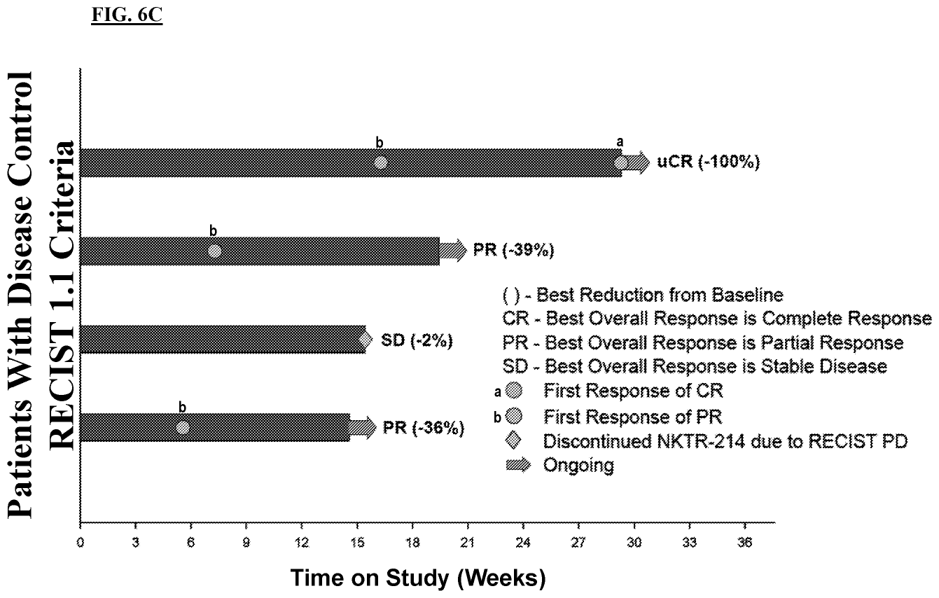

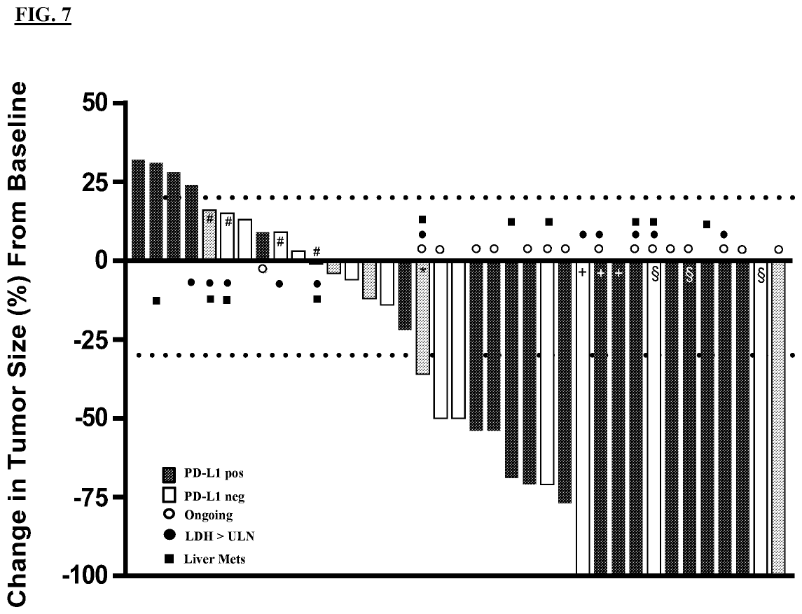

For NSCLC second line (2L) patients, the best overall response by RECIST was observed in 3/4 (75%) of patients, with 3/4 (75%) experiencing DCR (FIGS. 6A-6B). The median time to response was 1.7 months. Only one NSCLC 1L patient was enrolled in the study, and this patient experienced stable disease (FIG. 6A). All NSCLC patients were PD-L1 negative. Of the 4 NSCLC 1L/2L patients that exhibited DCR, 1 patient exhibited a complete response, 2 patents exhibited a partial response, and 1 patient exhibited stable disease (FIG. 6C).

The best overall response for each condition are summarized in Table 3.

TABLE 3
Best overall response by RECIST 1.1.

| | | 1L RCC | | | | |
|---|---|---|---|---|---|---|
| | 1L | | ≥2 scans | 2L | 1L | 2L |
| Patients | Melanoma | ≥1 scan | or PD** | RCC | NSCLC | NSCLC |
| Total Evaluable | 11 | 13 | 10 | 7 | 1 | 4 |
| ORR (CR + PR) | 7 (64%) | 6 (46%) | 6 (60%) | 1 (14%) | 0 (0) | 3 (75%) |
| CR | 2⁺ (18%) | 1^{#} (8%) | 1^{#} (10%) | 0 | 0 | 1^{#} (25%) |
| PR | 5 (45%) | 5 (38%) | 5 (50%) | 1 (14%) | 0 | 2 (50%) |
| SD | 3 (27%) | 5 (38%) | 2 (20%) | 6 (86%) | 1 (100%) | 0 |
| DCR | 10 (91%) | 11 (85%) | 8 (80%) | 7 (100%) | 1 (100%) | 3 (75%) |
| (CR + PR + SD) | | | | | | |
| PD | 1 | 2 | 2 | 0 | 0 | 1 |

CR, complete response;
DCR, disease control rate;
ORR, objective response rate;
PR, partial response;
PD, progressive disease;
SD, stable disease;
⁺CR is waiting to be confirmed for 1 of 2 patients with CR;
^{#}PR for patient confirmed. CR is waiting to be confirmed;
**Patients with at least 2 post-baseline scans or progressed on 1st post-baseline scan.

There were no study discontinuations due to treatment-related adverse events (AEs), and no treatment related deaths. Treatment-related AEs are summarized in Table 4.

TABLE 4
Treatment-related adverse events.

| | | CD-122- | CD-122- | CD-122- | CD-122- | CD-122- |
|---|---|---|---|---|---|---|
| | | biased agonist | biased agonist | biased agonist | biased agonist | biased agonist |
| | | 0.006 q3w + | 0.006 q3w + | 0.006 q2w + | 0.003 q2w + | 0.009 q3w + |
| Preferred | Total | Nivo 360 | Nivo 240 | Nivo 240 | Nivo 240 | Nivo 360 |
| Term^{[1]} | (N = 38) | (N = 25) | (N = 4) | (N = 3) | (N = 3) | (N = 3) |
| Grade 3 or 4 | 4 (10.5%) | 1 (4.0%) | 1 (25.0%) | 0 | 0 | 2 (66.7%) |
| Acidosis | 1 (2.6%) | 0 | 0 | 0 | 0 | 1 (33.3%)⋄ |
| Arthralgia | 1 (2.6%) | 0 | 1 (25.0%) | 0 | 0 | 0 |
| Diarrhea | 1 (2.6%) | 0 | 0 | 0 | 0 | 1 (33.3%)⋄ |
| Hyperglycemia | 1 (2.6%) | 0 | 0 | 0 | 0 | 1 (33.3%)⋄ |
| Hyperthyroidism | 1 (2.6%) | 0 | 0 | 0 | 0 | 1 (33.3%)⋄ |
| Hypotension | 1 (2.6%) | 1 (4.0%) | 0 | 0 | 0 | 0 |
| Syncope | 1 (2.6%) | 0 | 0 | 0 | 0 | 1 (33.3%) |
| Thyroiditis | 1 (2.6%) | 1 (4.0%) | 0 | 0 | 0 | 0 |

Grade 1&2 (>25%)

| | | | | | | |
|---|---|---|---|---|---|---|
| Fatigue | 28 (73.7%) | 17 (68.0%) | 4 (100.0%) | 2 (66.7%) | 3 (100.0%) | 2 (66.7%) |
| Flu Like | 26 (68.4%) | 15 (60.0%) | 3 (75.0%) | 3 (100.0%) | 2 (66.7%) | 3 (100.0%) |
| Symptoms** | | | | | | |
| Rash* | 23 (60.5%) | 13 (52.0%) | 4 (100.0%) | 1 (33.3%) | 2 (66.7%) | 3 (100.0%) |
| Pruritus | 16 (42.1%) | 8 (32.0%) | 2 (50.0%) | 2 (66.7%) | 2 (66.7%) | 2 (66.7%) |
| Headache | 14 (36.8%) | 8 (32.0%) | 3 (75.0%) | 1 (33.3%) | 1 (33.3%) | 1 (33.3%) |
| Nausea | 14 (36.8%) | 8 (32.0%) | 3 (75.0%) | 1 (33.3%) | 0 | 2 (66.7%) |
| Diarrhea | 12 (31.6%) | 8 (32.0%) | 2 (50.0%) | 0 | 1 (33.3%) | 1 (33.3%) |
| Arthralgia | 11 (28.9%) | 6 (24.0%) | 3 (75.0%) | 1 (33.3%) | 0 | 1 (33.3%) |
| Decreased | 10 (26.3%) | 3 (12.0%) | 3 (75.0%) | 2 (66.7%) | 0 | 2 (66.7%) |
| Appetite | | | | | | |

^{[1]}Patients are only counted once under each preferred term using highest grade;
*Rash includes the following MedDRA preferred terms: Rash, rash erythematous, rash macular and rash maculo-popular;
**Flu-like symptoms includes the following MedDRA preferred terms: influenza-like illness, pyrexia, and chills.
⋄AEs occurred in same patient, patient was dose reduced to CD-122-biased agonist 0.003 mg/kg + nivo 360 mg q3w and patient continues on treatment with ongoing confirmed PR.

CD-122-biased agonist plus nivolumab is a novel combination of immuno-oncology agents with differentiated and complementary mechanisms of immune activation. Efficacy results demonstrate important clinical activity in both PD-L1 negative and positive patients. All patients with responses continue on treatment. Few patients experienced rapid progression on treatment: melanoma first line: ORR 64% (2 CR, 5 PR), DCR 91%, mTTR 1.7 mos; RCC first line: ORR 46% (1 CR, 5 PR), DCR 85%, mTTR 1.9 mos; NSCLC second line (PD-L1 Negative): ORR 75% (1 CR, 2 PR), DCR 75%, mTTR 1.7 mos.

CD-122-biased agonist plus nivolumab is safe and tolerable and can be administered as a convenient, outpatient regimen. No study discontinuations due to TRAEs and no treatment related deaths. CD-122-biased agonist did not increase the risk for irAEs associated with nivolumab.

RP2D was established as CD-122-biased agonist 0.006 mg/kg plus nivolumab 360 mg delivered intravenously once every three weeks.

### Example 2

An open-label, phase 1/2 study is ongoing, investigating a CD-122-biased agonist in combination with nivolumab in patients with advanced cancers, including melanoma, Renal Cell Carcinoma (RCC), Non-small-cell lung carcinoma (NSCLC), triple negative breast cancer (TNBC), and urothelial carcinoma (UC). CD-122-biased agonist monotherapy increases newly proliferative CD8+ T cells in tumors and increases cell surface PD-1 and PD-L1 expression, demonstrating a potentially synergistic mechanism with anti-PD-1 therapy.

During P1 dose escalation, patients received 0.003 mg/kg, 0.006 mg/kg, or 0.009 mg/kg CD-122-biased agonist combined with 240 mg or 360 mg nivolumab, administered intravenously as outpatient one time every two or three weeks. During P2 expansion, RP2D of 0.006 mg/kg CD-122-biased agonist combined with 360 mg nivolumab was administered concurrently once every three weeks. Response was assessed every eight weeks by RECIST v1.1. Matched tumor samples were evaluated for changes from baseline in immune cell populations, gene expression, and T cell receptor repertoire. Tumor baseline PD-L1 protein expression was assessed by IHC assay using an anti-PD-L1 antibody (28-8).

One hundred and sixty two patients (P1, n=38; P2, n=124) were evaluable for safety. The most common treatment related adverse events (TRAEs) of all grades at RP2D (>25%) in patients were flu-like symptoms (63%), fatigue (39%), rash (38%), and pruritis (30%). Grade 3+ TRAEs at RP2D were 11%. No patients discontinued treatment due to TRAEs; and no treatment related deaths occurred at any dose.

A total of sixty immune-oncology (TO) treatment naïve Stage IV patients (P1, n=30; P2, n=30) were efficacy evaluable (>1 scan) (twenty three melanoma, twenty four RCC, six NSCLC, four UC, and three TNBC patients). Twenty two of thirty P2 patients had only one scan.

Overall response rate (CR+PR) and DCR (CR+PR+SD) in twenty three melanoma (1L) patients was 52% and 78%. Eighteen of twenty three MEL patients had known PD-L1 status. Overall response rate was 5/9 (56%) for PD-L1(+) patients and 4/9 (44/%) for PD-L1)(−) baseline patients. Overall response rate and DCR in twenty four RCC (1L) patients was 54% and 79%, respectively. Twenty of twenty four RCC patients had known PD-L1 status. Overall response rate was 4/7 (57%) for PD-L1(+) patients and 7/13 (54%) for PD-L1(−) patients. Overall response rate and DCR in six NSCLC (1-2L) patients was 50% and 67%, respectively. Five out of six patients had known PD-L1 status. Overall response rate was 3/5 (60%) in PD-L1(−) patients. Overall response rate and DCR in four UC (1L) was 75% and 100%, respectively. Overall response rate and DCR in three TNBC (1-2L) patients was 33%.

In sixty evaluable patients, thirty two of thirty two responses are ongoing (0.3+ to 12.0+ months) with forty five of sixty patients still on treatment.

Combination therapy with CD-122-biased agonist and nivolumab was well-tolerated with no treatment-related adverse event (TRAE) discontinuations. Preliminary efficacy shows encouraging ORR/DCR with responses observed in five of five tumor types treated.

### Example 3

In patients with melanoma, low levels of tumor-infiltrating lymphocytes and low/absent PD-L1 expression limit response to anti-PD-1/anti-PD-L1 therapies. Monotherapy using a CD-122-biased agonist (IL-2Rβγ-biased cytokine) stimulates proliferation and elevation of lymphocytes in blood and tumor and increases PD-1/PD-L1 expression. This example presents data on the impact of the CD-122-biased agonist and nivolumab on the systemic immune system and local tumor microenvironment.

Study Design

In an ongoing clinical trial, forty-one patients were enrolled, having locally advanced or metastatic solid melanoma (with known BRAF status), with measurable disease per RECIST v1.1, an ECOG score of PS 0-1, adequate organ function, and a fresh biopsy and archival tissue. Subjects were administered 0.006 mg/kg body weight of the CD-122-biased agonist once every three weeks plus a flat dose of 360 mg nivolumab once every three weeks as a first line therapy.

The primary endpoints include safety and tolerability per CTCAEv4.03; objective response rate (ORR) per RECIST v1.1, assessed once about every eight weeks; and efficacy, defined as patients with at least one post baseline scan. Secondary endpoints included BOR, duration of response (DOR), progression free survival (PFS), clinical benefit rate, mTTR, overall survival (OS), and pharmacokinetic (PK) data. In addition, biomarker endpoints further included absolute lymphocyte count, eosinophils, and blood immuno-phenotyping. Baseline and on-treatment biopsies (at week three) were collected in patients, when clinically feasible.

TABLE 5
Patient Demographics and Disease Characteristics

| | Total | |
|---|---|---|
| | (n = 41) | |
| Sex | | |
| Female | 17 | (41.5%) |
| Male | 24 | (58.5%) |
| Age (years) | | |
| Median (Range) | 63 | (22-80) |
| ECOG Performance Status | | |
| 0 | 32 | (78.0%) |
| 1 | 9 | (22.0%) |
| PD-L1 status* | | |
| Positive ≥1% | 21 | (51.2%) |
| Negative <1% | 15 | (36.6%) |
| Unknown | 5 | (12.2) |
| BRAF status | | |
| Mutant (V600E, V600K) | 15 | (36.6%) |
| Wild-Type | 25 | (61.0%) |
| Unknown | 1 | (2.4%) |
| LDH‡ | | |
| Normal | 29 | (70.7%) |
| Elevated >ULN# | 12 | (29.3%) |
| Stage (7th edition AJCC) | | |
| M0 | 0 | (0%) |
| M1a | 6 | (14.6%) |
| M1b | 18 | (43.9%) |
| M1c | 17 | (41%) |
| Liver metastases | | |
| Yes | 11 | (26.8%) |
| No | 30 | (73.2%) |

Demographics of biomarker subgroup are representative of overall population;
*PD-L1 status determined by 28-8 diagnostic on fresh or archival tumor, or investigator reported;
‡Based on maximum value prior to dosing;
#8 patients with >2X ULN and 4 patients with >3X ULN

All patients were evaluable for blood cell analysis, with thirty-eight patients evaluable for efficacy (>1 follow-up scan). Tumor biopsies were analyzed using multispectral IHC, gene expression, and TCR sequencing. Flow cytometry and hematology were used to evaluate blood cells. PD-L1 expression was evaluated using the DAKO 28-8 PharmDx Assay.

Results

Of the 38 evaluable patients treated with a combination of the CD-122-biased agonist and Nivolumab, 12 subjects (32%) experienced a 100% reduction of target lesions, and 9 subjects (24%) experienced complete responses (FIG. 7). The disease control rate (DCR; complete response (CR)+partial response (PR)+stable disease (SD)) was 76% (29/38). Subjects with PD-L1-negative tumors experienced at least a partial response rate of 43% (6/14); and 68% (13/19) of subjects with PD-L1-positive tumors experienced at least a partial response (20% (1/5) subjects having a tumor of unknown PD-L1 status experienced a 100% reduction in target lesion size). In addition, 45% (5/11) of subjects having LDH levels greater than or equal to two-times ULN had at least a partial response, as did 50% (5/10) of subjects with liver metastasis. A high level of concordance between blinded independent central review (BICR) (ORR of 50% (19/38)) and investigator assessed radiologic review was observed (ORR of 53% (20/38)).

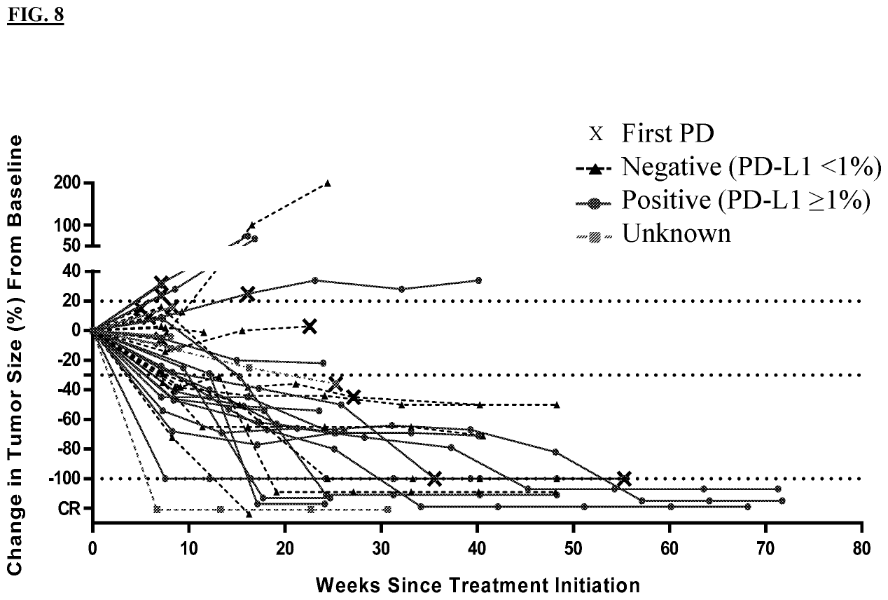

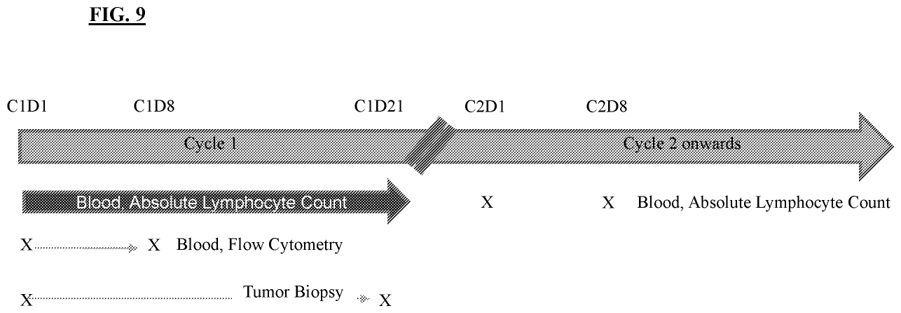

The median time to response was 2 months, with the median time of follow-up being 7.2 months (FIG. 8). The median duration of response was not reached (2.6, 16.6+), and the percent of patients still responding was 17 (85%). The median percent reduction from baseline was −50%.

The combination of nivolumab and the CD-122-biased agonist was well tolerated and can be administered in an outpatient setting. The tolerability profile in the melanoma cohort is consistent with the overall population in the study. There were no Grade 3 or higher cytokine related AEs, and there was a decreased frequency of observed AEs with continuous dosing. Cytokine related AE's decrease with subsequent cycles of treatment, all of which were low grade. AEs were easily managed with nonsteroidal anti-inflammatory drugs (NSAIDs) and over the counter (OTC) medications. No dose delays or dose reductions were required, and no subjects discontinued the study due to cytokine-related AEs. Hydration guidelines were effective, and no subjects experienced hypotension greater than or equal to Grade 3. The pro-drug design of the CD-122-biased agonist accounts for lower frequency of cytokine-related AEs compared to high dose IL-2.

Biomarkers

Activation of the IL-2 receptor pathway was demonstrated by various means: lymphocyte analysis in blood, immunophenotype analysis by flow cytometry of lymphocytes in blood, cellular analysis of tumor biopsy using immunofluorescence and IHC, gene expression of tumor biopsy using EdgeSeq, and TCR repertoire analysis using immunoSEQ. A schematic of the biomarker methodology is provided in FIG. 9. All patients were evaluated for absolute lymphocyte counts at all cycles. Blood for flow cytometry and tumor biopsy were collected from approximately 10 patients per tumor type at Cycle 1.

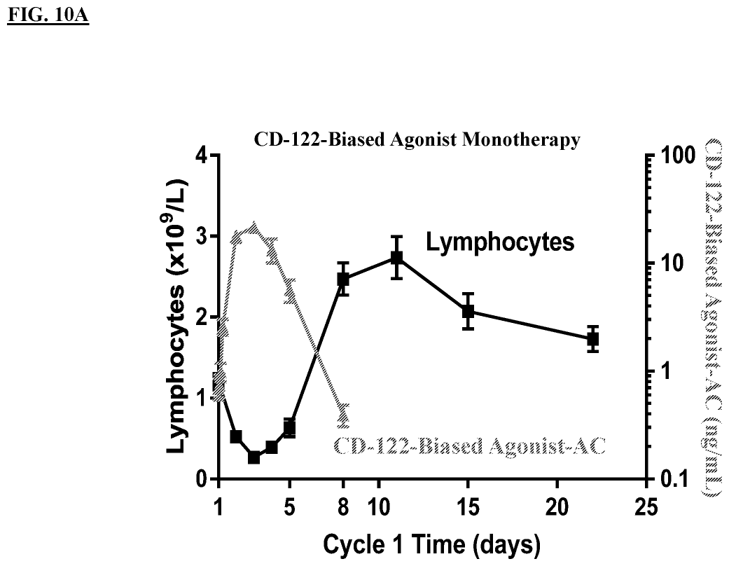

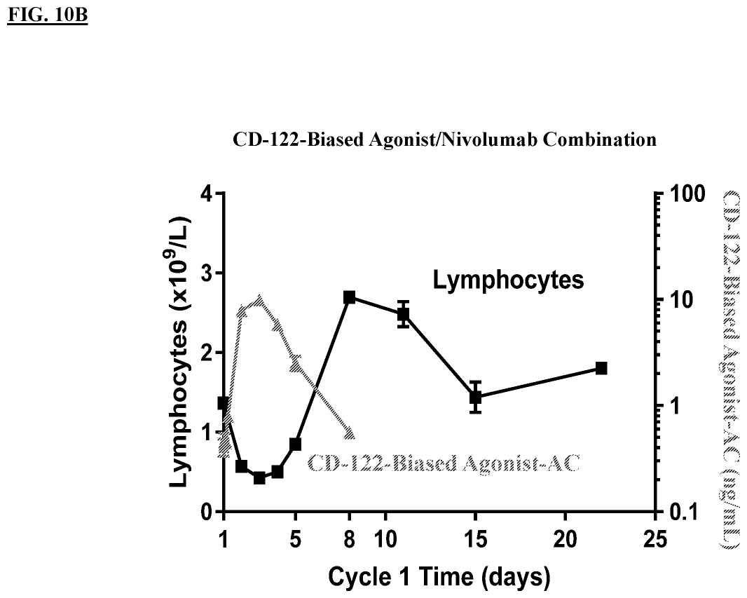

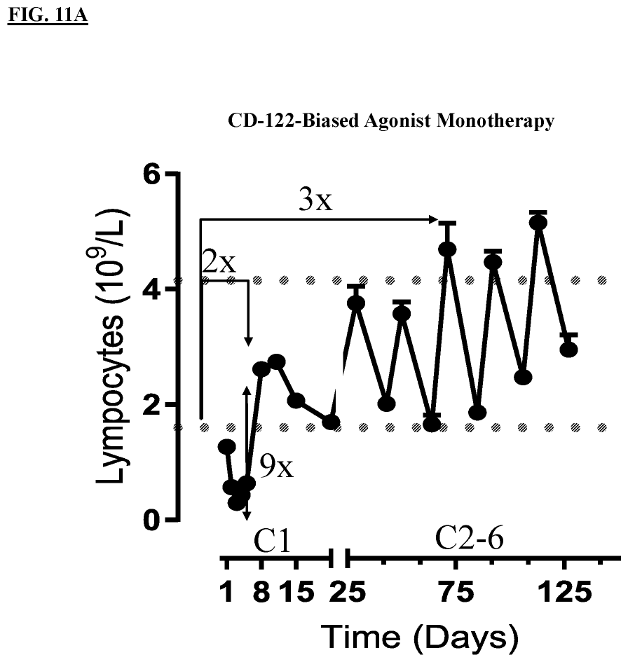

Transition of the CD-122-biased agonist prodrug to the active drug correlates with the number of lymphocytes in blood (FIGS. 10A-10B). The CD-122-biased agonist prodrug releases active cytokine (AC) over time. At days 2-4 post administration, the peak level of the CD-122-biased agonist AC coincides with transient lymphopenia (FIGS. 10A-10B). At days 8-10, transient lymphocytosis and the presence of proliferating (Ki67+) cells [not shown] are observed as the CD-122-biased agonist AC clears circulation (FIGS. 10A-10B). Lymphocyte effects are driven by the CD-122-biased agonist since effects were observed with CD-122-biased agonist monotherapy (FIG. 10A), with little contribution from nivolumab (FIG. 10B).

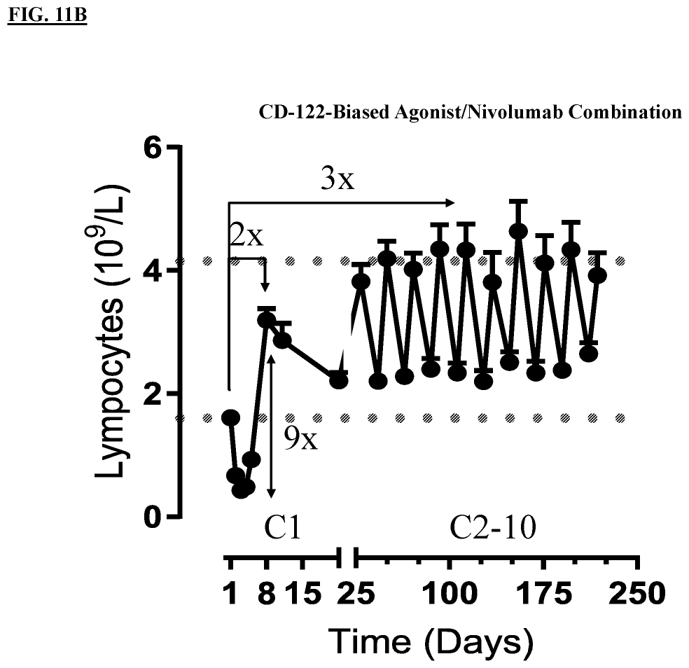

The CD-122-biased agonist was further observed to drive continuous mobilization of lymphocytes after every cycle, both as a monotherapy (FIG. 11A) and in combination with nivolumab (FIG. 11B). The CD-122-biased agonist provides rapid activation of the immune system. The effect of lymphocyte mobilization is consistent and maintained with successive treatment cycles. These lymphocyte effects are driven by the CD-122-biased agonist because the effects were observed with the CD-122-biased agonist monotherapy (FIG. 11A), with little contribution from Nivolumab (FIG. 11B).

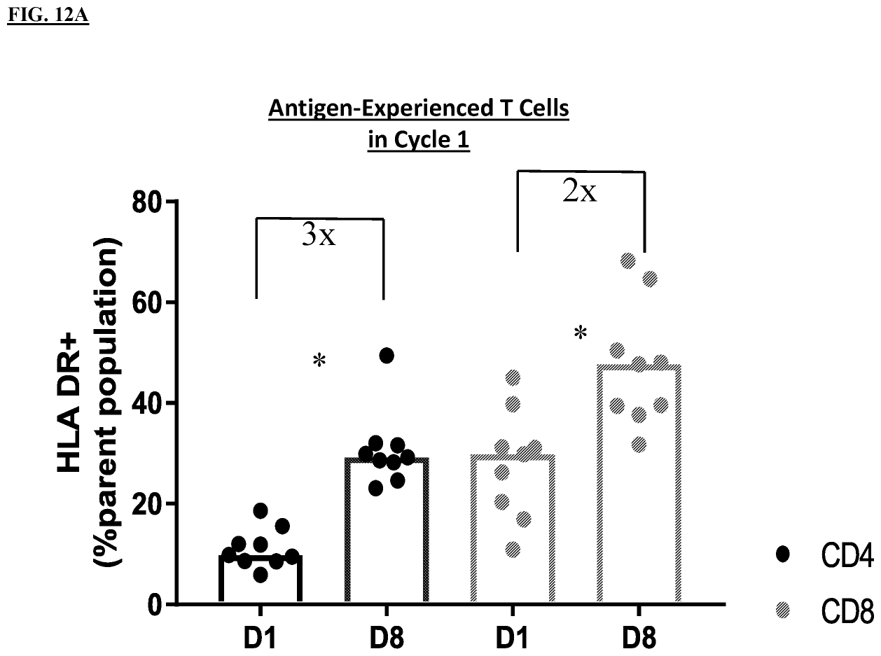

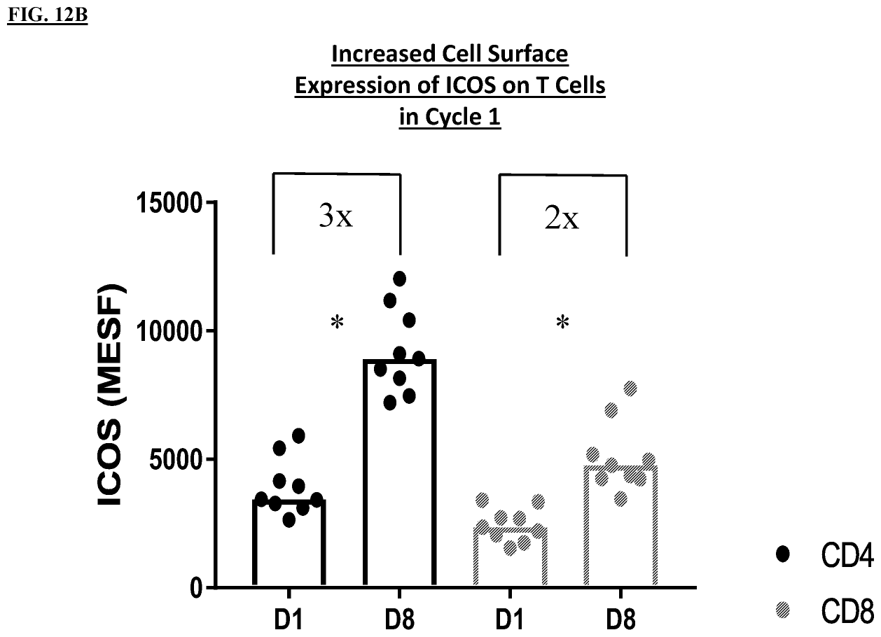

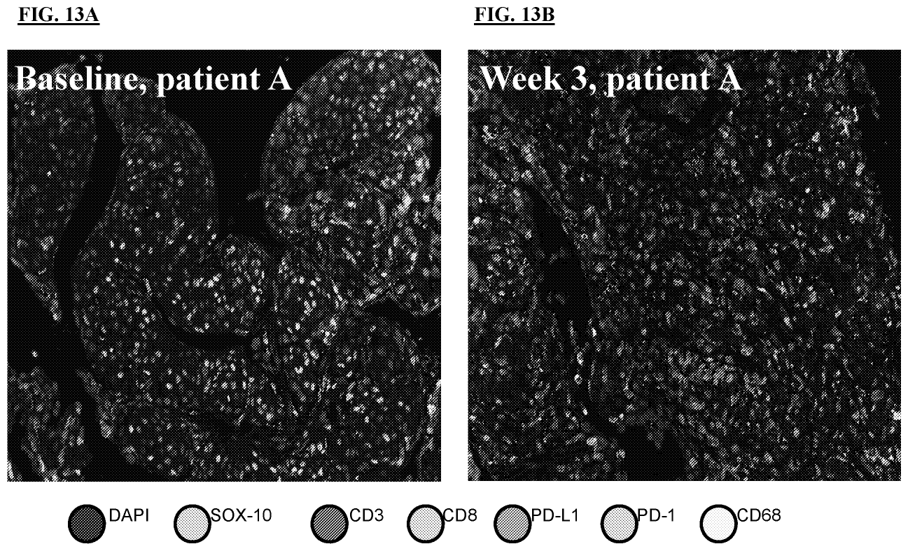

Immune monitoring of blood revealed clear activation of the IL-2 pathway after administration of the CD-122-biased agonist and nivolumab. Lymphocyte numbers increased nine-fold (N=41) from nadir, reaching their peak seven days post-dose. Lymphocyte numbers maintained that magnitude of increase after each cycle. The proportion of proliferating (Ki67+, N=12) CD4+, CD8+ and NK cells increased thirteen-fold, twenty-fold, and six-fold over baseline, respectively. Similar immune activation was reported with CD-122-biased agonist single agent (eight-fold, eight-fold, and seven-fold over baseline, respectively). Immune cells demonstrated an antigen-experienced phenotype with increased proportion of HLA-DR expression on CD4+, CD8+ and NK cells three-fold, two-fold, and six-fold over baseline, respectively (FIG. 12A; NK cell date not shown). ICOS levels increased three-fold on CD4+ T cells and two-fold on CD8+ T cells FIG. 12B). The ICOS increase was also observed following CD-122-biased agonist monotherapy (data not shown).

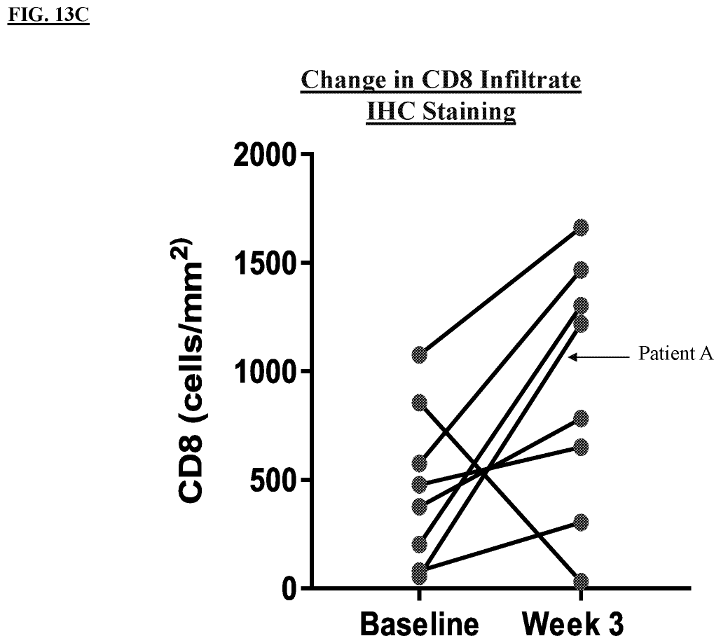

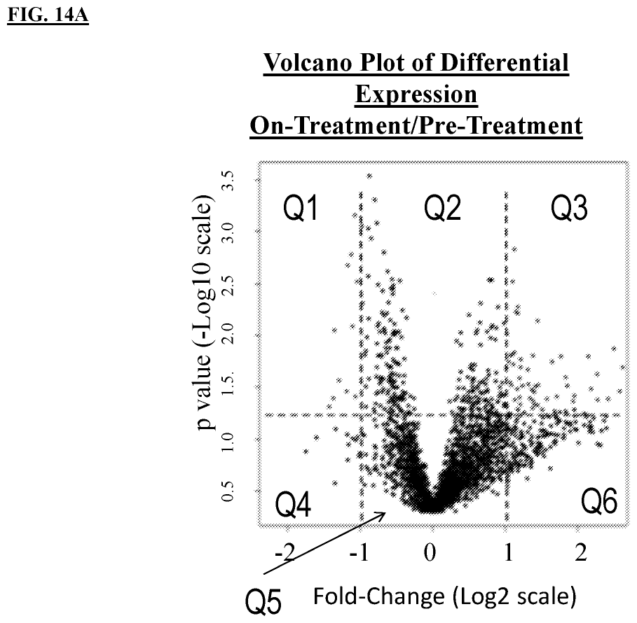

Serial tumor biopsies (n=12) demonstrated local effects on the tumor microenvironment including elevated expression of PD-L1 on the tumor (patients converted from PD-L1 negative to positive) (FIGS. 13A-13B), increased total numbers of CD8 infiltrate (FIG. 13C), and increased proportion of proliferating cells all ranging from six- to seventeen-fold over baseline (data not shown). There was good concordance between immunofluorescence and IHC methods.

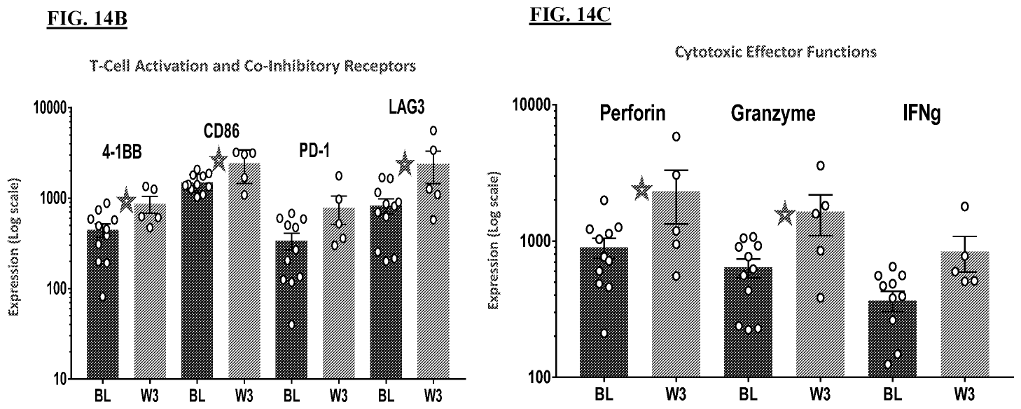

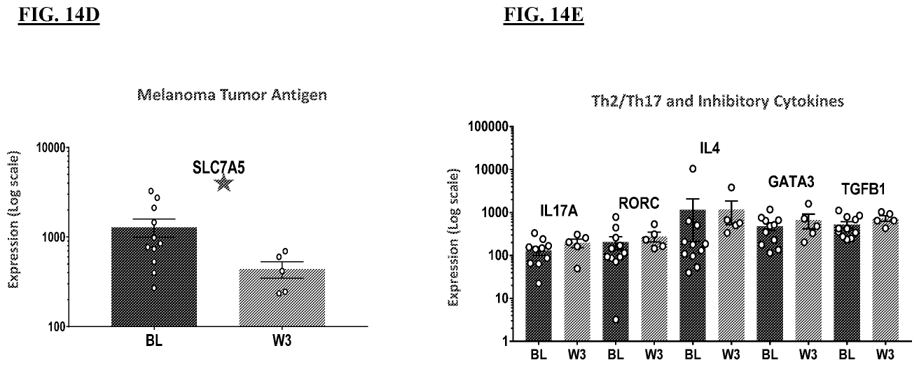

The combination of the CD-122-biased agonist and nivolumab promotes favorable anti-tumor gene expression changes and antigen reduction in the tumor. FIG. 14A provides a volcano plot of the differential expression on-treatment v. pre-treatment. Following treatment, intratumoral gene expression analyses showed elevations in networks associated with the CD-122-biased agonist mechanism of action, including induction of a Type II interferon gene signature. Genes encoding cell activation and co-inhibitory receptors (FIG. 14B; 4-1BB, CD86, PD-1, and LAG3) and genes encoding proteins with cytotoxic effector functions (FIG. 14C; perforin, granzyme, and IFNγ) were found to increase in expression at week 3 relative to baseline. In addition, expression of the melanoma tumor antigen SLC7A5 was found to decrease at week 3 relative to baseline (FIG. 14D). Little or no difference was observed in the expression Th2/TH17 and inhibitory cytokines (IL17A, RORC, IL4, GATA3, and TGFB1) at week 3 relative to baseline (FIG. 14E).

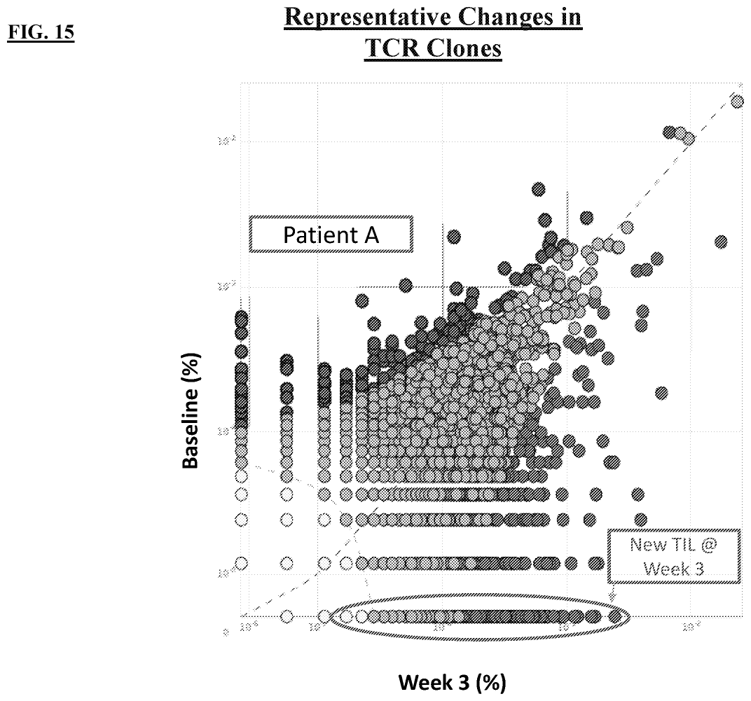

The combination of the CD-122-biased agonist and nivolumab drives new T cell clones into the tumor microenvironment. FIG. 15 shows the distribution of TCR clones at baseline and a week 3 for a select patient. All patients demonstrated new clones at week 3 that were not present at baseline. These results indicate that the combination therapy promotes new cell priming and T cell trafficking. In addition, a correlation was observed between baseline CD-8 tumor infiltrating lymphocytes and PD-L1 expression on best overall response

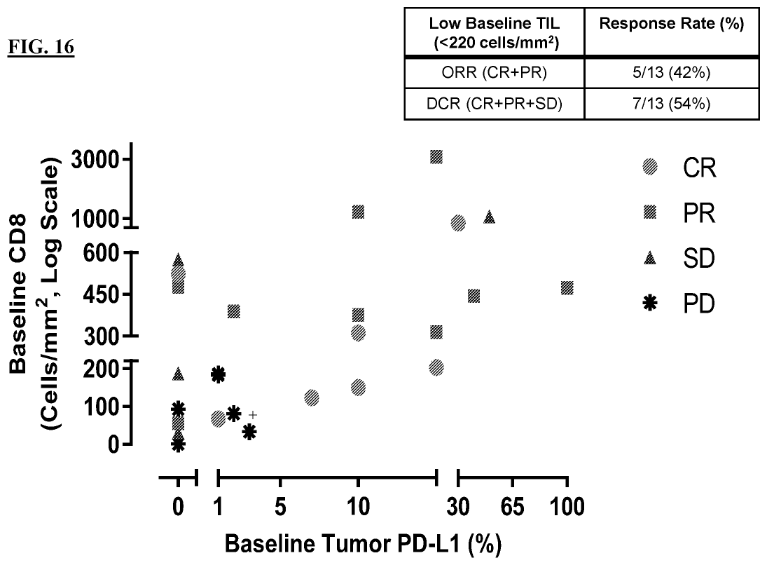

FIG. 16 shows the correlation between baseline CD8+ tumor infiltrating lymphocytes and PD-L1 expression on best overall response in melanoma patients following first line treatment with a combination therapy comprising a CD-122-biased agonist and nivolumab. Circles indicate complete response (CR), squares indicate partial response (PR), triangles indicate stable disease (SD), and asterisks indicate progressive disease (PD). Baseline tumor biopsies were evaluated by immunohistochemistry for CD8 cell counts (N=26), and PD-L1 expression (N=26) using the 28-8 method, or tumor mutation burden (TMB, N=12) using the Foundation TMB method. 42% (5/13) of patients with low baseline TIL had an ORR (CR or PR), and 54% (7/13) of subjects with low baseline TIL had DCR (CR, PR, or SD). TMB was evaluated for twelve patients with no apparent correlation between TMB and tumor reduction (data not shown).

Clear activation of the IL-2 pathway demonstrated by increase in absolute lymphocyte count with activated and proliferating CD4, CD8, and NK cells in blood. Serial tumor biopsies demonstrated beneficial effects on the tumor microenvironment including elevated expression of PD-L1, increased total numbers and proliferating of CD8 infiltrate and increased gene expression networks associated with T cell infiltrate and tumor killing. TCR repertoire analysis demonstrates the presence of newly trafficked clonal infiltrates after treatment with the CD-122-biased agonist plus nivolumab. Based on these data, the CD-122-biased agonist is a robust agonist of the IL-2 pathway and together with nivolumab promotes systemic and local immune activation for significant clinical activity. A global Phase 3 trial in treatment-naïve advanced melanoma patients of the CD-122-biased agonist plus nivolumab versus nivolumab (1:1) will be conducted.

### Example 4

Standard of care for treatment-naïve unresectable or metastatic MEL consists of checkpoint immunotherapy, including nivolumab. However, up to 55% of patients do not respond on nivolumab. IL-2 is a cytokine that has been validated as a cancer therapy, and has demonstrated pleiotropic effects on the immune system. A CD-122-biased agonist was designed to provide sustained signaling through the IL-2 βγ receptor to preferentially activate and expand effector CD8+ T and NK cells over T regulatory cells (Hurwitz M E et al. ASCO GU 2017). In a phase 1 trial, the CD-122-biased agonist monotherapy was well tolerated (Hurwitz M E et al. ASCO GU 2017). In the dose expansion phase of the phase 1/2 PIVOT-02 trial, the CD-122-biased agonist plus nivolumab was also well tolerated at the recommended phase 2 dose (RP2D; CD-122-biased agonist 0.006 mg/kg IV once every three weeks plus nivolumab 360 mg IV once every three weeks), and many melanoma patients on first-line CD-122-biased agonist plus nivolumab at RP2D achieved an overall response rate (ORR) of 85% and disease control rate of 71% (Diab A et al. ASCO 2018). Here, we present the design of the first phase 3 trial in the CD-122-biased agonist plus nivolumab development program.

Methods

This phase 3, randomized, open-label study aims to evaluate the effectiveness, safety, and tolerability of CD-122-biased agonist plus nivolumab. Eligible subjects are at least 12 years old with histologically confirmed stage III or stage IV melanoma and ECOG performance status (PS)≤1 or Lansky PS ≥80%. Subjects are ineligible if they have active brain or leptomeningeal metastases, uveal melanoma, or a recurrence within 6 months of completing adjuvant treatment. Subjects will be stratified by PD-L1 status, BRAF mutation status, and MO/M1 any (0) vs M1 any (1). Subjects will be randomized to receive a CD-122-biased agonist plus nivolumab RP2D or nivolumab 360 mg IV once every three weeks up to 24 months, RECIST 1.1 progression, or unacceptable toxicity (estimated N=764). Primary endpoints are ORR and progression-free survival (PFS) by blinded independent central review (BICR) and overall survival (OS). Secondary endpoints include ORR and PFS by investigator and BICR in biomarker population, OS in biomarker population, and safety. Additional endpoints include pharmacokinetics and quality of life assessment.

### Example 5

In on-going study, subjects having advanced and/or metastatic urothelial carcinoma (UC) were administered 0.006 mg/kg of a CD-122-biased agonist intravenously plus 360 mg of nivolumab intravenously once every three weeks as a first line therapy. Subjects were either ineligible to first line therapy with cisplatin or refused a standard of care therapy. Responses were assessed every eight weeks. Matched blood and tumor biopsies were evaluated for biomarkers including PD-L1 expression (assessed by Dako 28-8 PharmaDx IHC), wherein PD-L1-positive is defined as ≥1% tumor cell staining.

Thirty-four subjects have received one or more doses of treatment (cisplatin ineligible [n=22]; refused SOC [n=12]). The median age of the subjects is 70. Of the thirty-four subjects, twenty-three were efficacy evaluable (defined per protocol as having one or more post-treatment scan), seven were pending a first scan, one subject was excluded for non-eligibility (no target lesion), and three subjects discontinued prior to first scan. Thresholds for efficacy were exceeded in all first-line advanced/metastatic UC cohorts under a pre-specified Fleming objective response rate (ORR) analysis. In the efficacy evaluable population, overall ORR was 48% (11/23; 95% CI 27—69%) with a 17% CR rate (4/23) and 70% (16/23) DCR. The ORR was 50% in PD-L1-negative subjects (5/10; 95% CI 19—81%) and 56% in PD-L1-positive subjects (5/9; 95% CI 21—86%). PD-L1 status was unknown in four efficacy-evaluable subjects. The most common treatment-related adverse events (TRAE; >30%) were fatigue (59%), pyrexia (38%), chills (32%), and flu-like symptoms (32%). Grade 3 or higher TRAEs occurred in 18% of subjects, and 8.8% of subjects discontinued due to TRAEs. No grade 4 or grade 5 TRAEs occurred. Twenty-two subjects had available baseline PD-L1 results (PD-L1-positive [n=11]; PD-L1 negative [n=11]). Ten of the eleven PD-L1-negative baseline samples had matched week 3 biopsies. Of these, 6/10 (60%) converted to PD-L1-positive at week 3.

Combination therapy with the CD-122-biased agonist and nivolumab showed encouraging clinical activity, including complete responses, and an acceptable preliminary safety profile in subjects with advanced/metastatic UC. Efficacy appears independent of PD-L1 status with a similar ORR in PD-L1-negative and PD-L1-positive tumors.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All publications, patents, and patent applications disclosed herein are incorporated by reference to the same extent as if each individual publication, patent or patent application was specifically and individually indicated to be incorporated by reference.

This application claims the benefit of U.S. Provisional Application Nos. 62/582,174, filed Nov. 6, 2017, 62/629,481, filed Feb. 12, 2018, and 62/712,814, filed Jul. 31, 2018, which are incorporated by reference herein in their entireties.

1. A method of treating a subject afflicted with a tumor derived from a melanoma comprising administering to the subject an antibody that binds specifically to a Programmed Death-1 (PD-1) receptor and inhibits PD-1 activity (“anti-PD-1 antibody”) and a CD-122-biased agonist, wherein the anti-PD-1 antibody is administered at a flat dose once about every 1, 2, 3, or 4 weeks.

2. The method of claim 1, wherein the CD-122-biased agonist comprises an interleukin-2 (IL-2) protein conjugated to a water-soluble polymer.

3. (canceled)

4. The method of claim 1, wherein the CD-122-biased agonist:
(a) interacts with an interleukin-2 receptor βγ (IL-2Rβγ) on the surface of a cell;
(b) interacts more strongly with an IL-2Rβγ on the surface of a cell than the CD-122-biased agonist interacts with an IL-2Rαβγ on the surface of the cell;
(c) promotes clonal expansion of NK cells, CD8+ cells, CD4+ helper T cells, or any combination thereof;
(d) does not promote clonal expansion of CD4+ Treg cells; or
(e) any combination of (a)-(d).

5. The method of claim 4, wherein the cell of (a) and/or (b) is selected from the group consisting of a natural killer (NK) cell, a CD4+ cell, a CD8+ cell, and any combination thereof.

6. The method of claim 1, wherein the CD-122-biased agonist comprises the following formula: 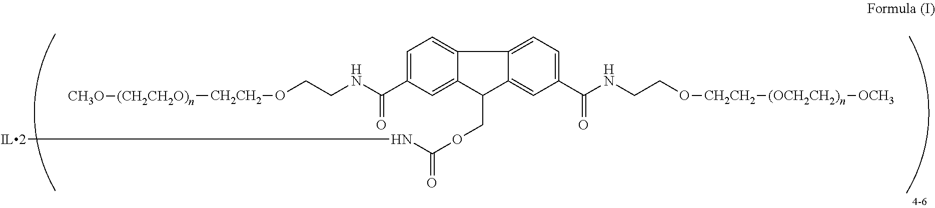
wherein each “n” is independently an integer from about 3 to about 1000.

7. The method of claim 1, wherein the administration of the CD-122-biased agonist:
(a) increases proliferation of tumor infiltrating lymphocytes (TILs) in the tumor as compared to the proliferation of TILs in the tumor prior to the administration;
(b) increases PD-1 expression on effector T cells in the subject as compared to the PD-1 expression on effector T cells prior to the administration; or
(c) both (a) and (b).

8. The method of claim 1, wherein the anti-PD-1 antibody comprises nivolumab.

9. The method of claim 1, wherein the anti-PD-1 antibody is administered at a flat dose of about 200, about 240, about 360, or about 480 mg once about every 2, 3, or 4 weeks.

10. The method of claim 1, wherein the CD-122-biased agonist is administered at a dose ranging from at least about 0.0001 mg/kg to at least about 0.1 mg/kg body weight once about every 1, 2, 3, or 4 weeks.

11. The method of claim 1, wherein the anti-PD-1 antibody is administered at a dose of about 360 mg every 3 weeks and the CD-122-biased agonist is administered at a dose of about 0.006 mg/kg body weight about every 3 weeks.

12. The method of claim 1, wherein
the anti-PD-1 antibody and the CD-122-biased agonist are administered concurrently in separate pharmaceutical compositions.

13. The method of claim 1, wherein the tumor comprises one or more cells that express PD-L1, PD-L2, or both.

14. The method of claim 1, wherein the subject received at least one prior chemotherapy treatment.

15. A kit for treating a subject afflicted with a cancer, the kit comprising:
(a) a dosage ranging from about 10 mg to about 600 mg of an anti-PD-1 antibody;
(b) a dosage ranging from about 0.0001 mg to about 0.1 mg of a CD-122-biased agonist;
(c) instructions for administering the anti-PD-1 antibody and the CD-122-biased agonist to a subject.

16. The method of claim 1, wherein the anti-PD-1 antibody is administered at a flat dose of about 240 mg once about every 2 weeks.

17. The method of claim 1, wherein the anti-PD-1 antibody is administered at a flat dose of about 360 mg once about every 3 weeks.

18. The method of claim 1, wherein the anti-PD-1 antibody is administered at a flat dose of about 480 mg once about every 4 weeks.

19. The method of claim 1, wherein the anti-PD-1 antibody comprises nivolumab, and wherein nivolumab is administered at a dose of about 360 mg every 3 weeks, and the CD-122-biased agonist is administered at a dose of about 0.006 mg/kg body weight about every 3 weeks.

20. The method of claim 1, wherein the anti-PD-1 antibody comprises pembrolizumab.

21. The method of claim 1, wherein the anti-PD-1 antibody and the CD-122-biased agonist are admixed as a single composition for concurrent administration.
